(19) United States
(12) Patent Application Publication
(10) Pub. No.: US 2025/0004683 A1
(43) Pub. Date: Jan. 2, 2025

(54) METHOD, APPARATUS AND COMPUTER PROGRAM PRODUCT FOR ALLEVIATING MOTION SICKNESS IN A USER VIEWING AN IMAGE ON A DISPLAY

(71) Applicants: Sony Group Corporation, Tokyo (JP); Sony Europe B.V., Weybridge (GB)

(72) Inventors: Renaud DI FRANCESCO, London (GB); Takeshi HORIE, Kawagawa (JP)

(21) Appl. No.: 18/711,580

(22) PCT Filed: Nov. 25, 2022
(86) PCT No.: PCT/GB2022/052992

§ 371 Date: May 18, 2024

(30) Foreign Application Priority Data
Dec. 23, 2021 (GB) 2118960.0

(51) Int. Cl. G06F 3/14; G06F 3/01; G09G 5/38; H04N 5/74

(52) U.S. Cl. CPC G06F 3/14; G06F 3/011; G09G 5/38; H04N 5/74; G09G 2340/0464

(57) ABSTRACT

The present disclosure provides a method of alleviating motion sickness in a user viewing an image on a display, the method comprising: displaying an image on a display device, the display device being a first distance from a user viewing the image on the display device; generating a virtual screen corresponding to the display device by projecting a line from the user and the display device to a second location, the distance of the second location from the user being greater than the first distance; detecting a movement of the user and the display device to a new location relative to the virtual screen; determining a new image location by projecting a line from the virtual screen location to the new location of the user; and displaying an image on the display device at the new image location for at least a portion of the image for which the new image location intersects with the display device.

### BACKGROUND

### Field of Disclosure

The present invention relates to a method, apparatus and computer program product for alleviating motion sickness in a user viewing an image on a display.

### Description of the Related Art

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present invention.

Display devices are used with a wide range of electronic devices in order to display information to a user. For example, a display device is found on personal electronic devices such as a mobile telephone device, a personal computer, a laptop computer, a tablet computer device and the like. The display device (such as a display screen) is used in order to display various kinds of images to the user. These images can correspond to certain information which should be provided to the user (e.g. information including certain messages or notifications). Alternatively, the images can relate to digital content such as film (movie), television programme, computer game or the like. As display devices are now required by such a wide range of electronic devices, the use of display devices in different environments, such as when travelling, has become more common. For example, a user may use a display device to watch digital content when walking to a destination, travelling in a vehicle such as a car, flying on a plane or the like. However, use of display device in these sorts of environments can lead to contradictory sensory information being provided to the user causing the user to experience a sense of motion sickness. This is because there can be a conflict between the sense of motion experienced by a user when travelling and the visual content the user sees on the display device. In some situations, this motion sickness can even prevent the display device from being used by the user. Accordingly, important information such as warning or messages may be missed by a user experiencing motion sickness.

Such problems are exacerbated in the case of autonomous or semi-autonomous vehicles, such as an autonomous or semi-autonomous car. Here, passengers in the autonomous or semi-autonomous car may be able to use a display device to view digital content while the car is operating in an autonomous mode. However, as the passengers have no control over the vehicle, they may experience motion sickness owing to the disconnect between their own sense of motion and their attention (e.g. what they look and listen to). Moreover, in such a vehicle, a designated passenger may be required to switch from being a passenger to becoming an active driver (e.g. if the traffic, context or situation requires it). In such a case, the passenger who becomes an active driver may be experiencing motion sickness at a time when they should be driving again. If the passenger is suffering from motion sickness, then it may be difficult for the passenger to take appropriate control of the car. This can, potentially, be dangerous for the car and other road users.

It is an aim of the present disclosure to address these issues, although the disclosure may solve other issues.

### SUMMARY

A brief summary about the present disclosure is provided hereinafter to provide basic understanding related to certain aspects of the present disclosure.

In a first aspect of the present disclosure, a method of alleviating motion sickness in a user viewing an image on a display is provided, the method comprising: displaying an image on a display device, the display device being a first distance from a user viewing the image on the display device; generating a virtual screen corresponding to the display device by projecting a line from the user and the display device to a second location, the distance of the second location from the user being greater than the first distance; detecting a movement of the user and the display device to a new location relative to the virtual screen; determining a new image location by projecting a line from the virtual screen location to the new location of the user; and displaying an image on the display device at the new image location for at least a portion of the image for which the new image location intersects with the display device.

In a second aspect of the present disclosure, an apparatus for alleviating motion sickness in a user viewing an image on a display is provided, the apparatus comprising circuitry configured to: display an image on a display device, the display device being a first distance from a user viewing the image on the display device; generate a virtual screen corresponding to the display device by projecting a line from the user and the display device to a second location, the distance of the second location from the user being greater than the first distance; detect a movement of the user and the display device to a new location relative to the virtual screen; determine a new image location by projecting a line from the virtual screen location to the new location of the user; and display an image on the display device at the new image location for at least a portion of the image for which the new image location intersects with the display device.

In a third aspect of the present disclosure, a computer program product comprising instructions which, when the instructions are implemented by a computer, cause the computer to perform a method of alleviating motion sickness in a user viewing an image on a display is provided, the method comprising: displaying an image on a display device, the display device being a first distance from a user viewing the image on the display device; generating a virtual screen corresponding to the display device by projecting a line from the user and the display device to a second location, the distance of the second location from the user being greater than the first distance; detecting a movement of the user and the display device to a new location relative to the virtual screen; determining a new image location by projecting a line from the virtual screen location to the new location of the user; and displaying an image on the display device at the new image location for at least a portion of the image for which the new image location intersects with the display device.

Other embodiments of the present disclosure are defined by the appended claims.

According to embodiments of the disclosure, a reduction in the conflict between the sensory information provided to a user when viewing an image on a display can be achieved which alleviates the sense of motion experience for the user. This enables display devices to be used more safely in a wide range of environments. The present disclosure is not particularly limited to these advantageous technical effects. Other technical effects may become apparent to the skilled person when reading the disclosure.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates an example apparatus in accordance with embodiments of the disclosure;

FIG. 2 illustrates an example situation of motion sickness in accordance with embodiments of the disclosure;

FIG. 3 illustrates an example configuration of an apparatus in accordance with embodiments of the disclosure;

FIG. 4 illustrates an example of generating a virtual screen in accordance with embodiments of the disclosure;

FIG. 5 illustrates an example of determining a new image location in accordance with embodiments of the disclosure;

FIG. 6 illustrates an example situation to which embodiments of the disclosure may be applied;

FIG. 7 illustrates an example situation to which embodiments of the disclosure may be applied;

FIG. 8 illustrates an example situation to which embodiments of the disclosure may be applied;

FIG. 9 illustrates an example method in accordance with embodiments of the present disclosure;

FIG. 10 illustrates an example configuration of an apparatus in accordance with embodiments of the disclosure;

FIG. 11 illustrates an example journey in accordance with embodiments of the disclosure;

FIG. 12 illustrates an example timeline in accordance with embodiments of the disclosure;

FIG. 13 illustrates an example of content with different characteristics in accordance with embodiments of the disclosure;

FIG. 14 illustrates an example timeline in accordance with embodiments of the disclosure;

FIG. 15 illustrates an example method in accordance with embodiments of the disclosure.

### DESCRIPTION OF THE EMBODIMENTS

Referring now to the drawings, wherein like reference numerals designate identical or corresponding parts throughout the several views.

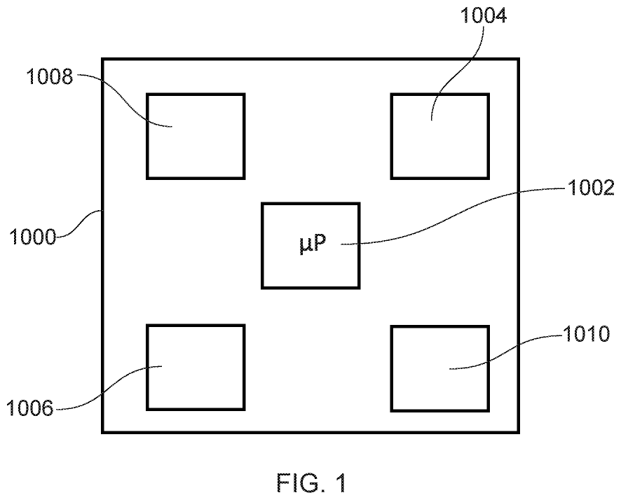

Referring to FIG. 1, an apparatus 1000 according to embodiments of the disclosure is shown. Typically, an apparatus 1000 according to embodiments of the disclosure is a computer device such as a personal computer or a terminal connected to a server. Indeed, in embodiments, the apparatus may also be a server. The apparatus 1000 is controlled using a microprocessor or other processing circuitry 1002. In some examples, the apparatus 1000 may be a portable computing device such as a mobile phone, laptop computer or tablet computing device or the like.

The processing circuitry 1002 may be a microprocessor carrying out computer instructions or may be an Application Specific Integrated Circuit. The computer instructions are stored on storage medium 1004 which maybe a magnetically readable medium, optically readable medium or solid state type circuitry. The storage medium 1004 may be integrated into the apparatus 1000 or may be separate to the apparatus 1000 and connected thereto using either a wired or wireless connection. The computer instructions may be embodied as computer software that contains computer readable code which, when loaded onto the processor circuitry 1002, configures the processor circuitry 1002 to perform a method according to embodiments of the disclosure.

Additionally, an optional user input device 1006 is shown connected to the processing circuitry 1002. The user input device 1006 may be a touch screen or may be a mouse or stylus type input device. The user input device 1006 may also be a keyboard or any combination of these devices.

A network connection 1008 may, optionally, be coupled to the processor circuitry 1002. The network connection 1008 may be a connection to a Local Area Network or a Wide Area Network such as the Internet or a Virtual Private Network or the like. The network connection 1008 may be connected to a server allowing the processor circuitry 1002 to communicate with another apparatus in order to obtain or provide relevant data. The network connection 1002 may be behind a firewall or some other form of network security.

Additionally, shown coupled to the processing circuitry 1002, is a display device 1010. The display device 1010, although shown integrated into the apparatus 1000, may additionally be separate to the apparatus 1000 and may be a monitor or some kind of device allowing the user to visualize the operation of the system. In addition, the display device 1010 may be a projector or some other device allowing relevant information generated by the apparatus 1000 to be viewed by the user or by a third party. The display device may also be any type of display screen (e.g. an LED screen, OLED screen or the like) or, alternatively, may be a headset such as a virtual reality (VR) or augmented reality (AR) display device. A display device in the present disclosure is therefore any device which allows relevant images and information to be viewed by the user or by a third party.

As explained in the Background, display devices are now found in a wide range of electronic devices which means that display devices are now being used in an increasingly wider range of environments and situations. These environments and situations may include use of the display device while travelling (i.e. moving from one place to another). However, motion sickness (including, but not limited to, symptoms of nausea and/or loss of balance) can occur in a user (i.e. any person) when they are travelling. Motion sickness often arises owing to a conflict of sensory information. That is, a person maintains their sense of balance using signals from many different sensory organs and parts of the body. This includes signals from the person's eyes and signals from their inner ears. When travelling, conflict between these sense can arise. Taking an example of travelling on a train, a person's inner ears may sense the movement of the train yet their eyes may not be able see any sign of this movement (as the inside of a train remains fixed or stationary relative to the user, for example). Therefore, a sense of motion sickness can arise owing to this conflict between the person's senses. In fact, viewing an image on a display device while travelling may increase the sense of motion sickness. This is because further conflict may arise from the images viewed on the display device and the person's other senses (such as the sense of motion from their inner ear). It can be difficult to use display devices in these environments and situations. However, the use of display devices is often desired even when travelling. This can be for safety critical information, navigation information, entertainment, or business purposes. As such, it is desired to reduce or alleviate motion sickness in a person when using a display device—particularly when using a display device while travelling.

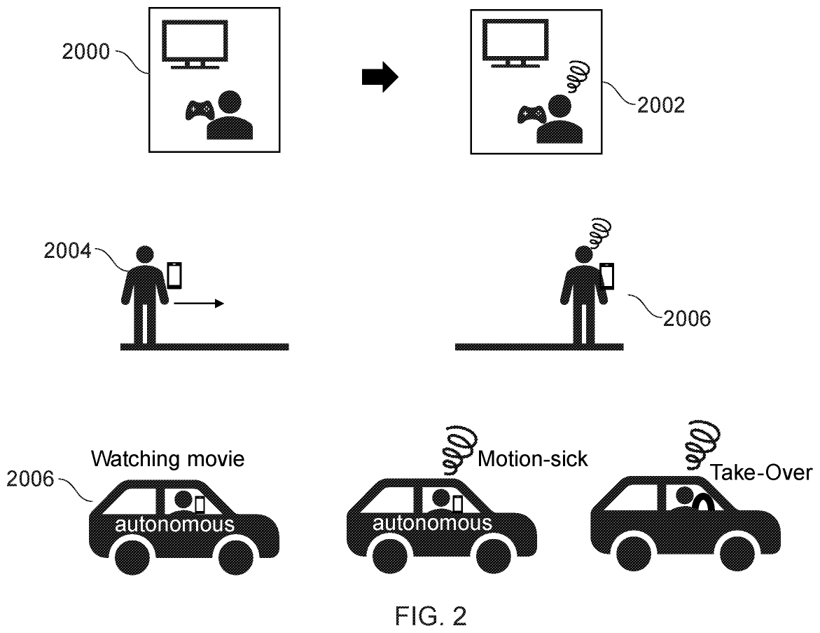

Referring now to FIG. 2 of the present disclosure, an example situation of motion sickness is illustrated.

Panel 2000 of FIG. 2 illustrates a situation where a user is playing a videogame. The user is watching the videogame on a display device (e.g. a computer screen) and is using a controller to provide input which causes a certain action to happen within the videogame. The user's eyes are therefore fixed on the display device. When the user is playing the videogame in a static location (such as when seated at home) the user may be able to play the videogame without any feeling of motion sickness arising. However, panel 2002 of FIG. 2 illustrates a situation where a user is playing a videogame while travelling on a vehicle such as a plane. Here, the user is watching the display device in the same manner as described for 2002. However, because the user is travelling on a plane, a feeling of motion sickness may arise. This is because the plane will undergo movement such as vibration, turbulence, changes of altitude, direction and/or acceleration for example. This movement may be sensed by the inner ear of the user travelling on the plane. However, the user's eyes—being fixed on the display device—will not be able to see any visual sign of this movement. Indeed, movement which is seen by the user (e.g. movement within the videogame) may even be in conflict with the sense of movement detected by the user's inner ear. This disparity between the user's senses may cause the user to experience motion sickness. Accordingly, the user may have to stop using the display device. While, in this example, the user is using the display device to play a videogame, the display device may also be used in order to provide important information or warnings to the user. As such, if the user has to spontaneously stop using the display device because of their motion sickness, important information and/or warnings may be missed. This can be dangerous.

Motion sickness may also arise when using a display device even if the user is not travelling in a vehicle. Panels 2004 and 2006 of FIG. 2 of the disclosure illustrate a situation where a user is using a display device, such as a display on a mobile telephone device, when walking. Here, the user is focusing on the display device of their mobile telephone. The user may be reading an important email from their employer, for example. As such, the user's eyes may not be able to see movement as they are intently fixed on the display device. However, because the user is walking along, their other senses may detect movement. A conflict between the user's senses can therefore arise leading to a sense of motion sickness. Accordingly, the user may have to stop using the display device. Moreover, if the user loses their sense of balance because of their motion sickness, they are more likely to fall over or bump into obstacles or other people while walking along. This can be dangerous.

In panel 2006 a third example situation is shown. Here, a user is travelling in an autonomous (or self-driving) car. The user may be a passenger and may, further, be a passenger who is designated driver of the car (i.e. a passenger who will become the active driver if required). In an autonomous or semi-autonomous car, the passenger may be able to perform certain actions such as watching a film (movie) on a display device while the car in in autonomous mode. However, it may be necessary for the passenger to take control of the car in certain situations (e.g. become the active driver). If the passenger has been watching a display device while the car is in autonomous mode, then the passenger may experience a sense of motion sickness at the time when they are required to become the active driver. That is, signals from the passenger's eyes which are fixed on the display device may contradict the signals from the passenger's other senses corresponding to the movement of the car as it drives along the road. The passenger may therefore have to stop watching the display device. Moreover, if the passenger has to suddenly take-over control of the car then the passenger may take over the control of the car while experiencing a sense of motion sickness. This can be dangerous.

### First Embodiment—Dynamic Adjustment

In view of the above (and also the reasons described in the Background of the disclosure) it is desired to alleviate the sense of motion sickness when viewing an image on a display device. Accordingly, an apparatus, method and computer program product for alleviating motion sickness in a user viewing an image on a display are provided in accordance with the embodiments of the disclosure.

### <Apparatus>

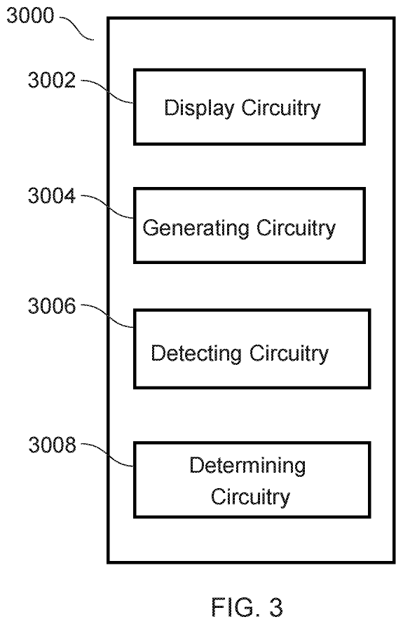

FIG. 3 of the present disclosure illustrates an example configuration of an apparatus in accordance with embodiments of the disclosure. The apparatus 3000 may be used in order to alleviate motion sickness of a user when viewing an image on a display.

The apparatus 3000 comprises a display circuitry 3002, a generating circuitry 3004, a detecting circuitry 3006 and a determining circuitry 3008.

The display circuitry 3002 may be implemented as processing circuitry configured to display an image on a display device, the display device being a first distance from a user viewing the image on the display device.

The generating circuitry 3004 may be configured to generate a virtual screen corresponding to the display device by projecting a line from the user and the display device to a second location, the distance of the second location from the user being greater than the first distance.

The detecting circuitry 3006 may be configured to detect a movement of the user and the display device to a new location relative to the virtual screen.

The determining circuitry 3008 may be configured to determine a new image location by projecting a line from the virtual screen location to a new location of the user.

Finally, the display circuitry may also be configured to display an image on the display device at the new image location for at least a portion of the image for which the new image location intersects with the display device.

In this manner, virtual screen which is generated by the apparatus 3000 emulates a physical screen (or other type of display device) located at a greater distance from the user than the actual display device. Moreover, motion of the user and the display device relative to this virtual screen is accounted for by corresponding movement of the image on the display device. Accordingly, conflict between the image seen by the user's eye and the movement of the user and the display device can be resolved which alleviates a sense of motion sickness.

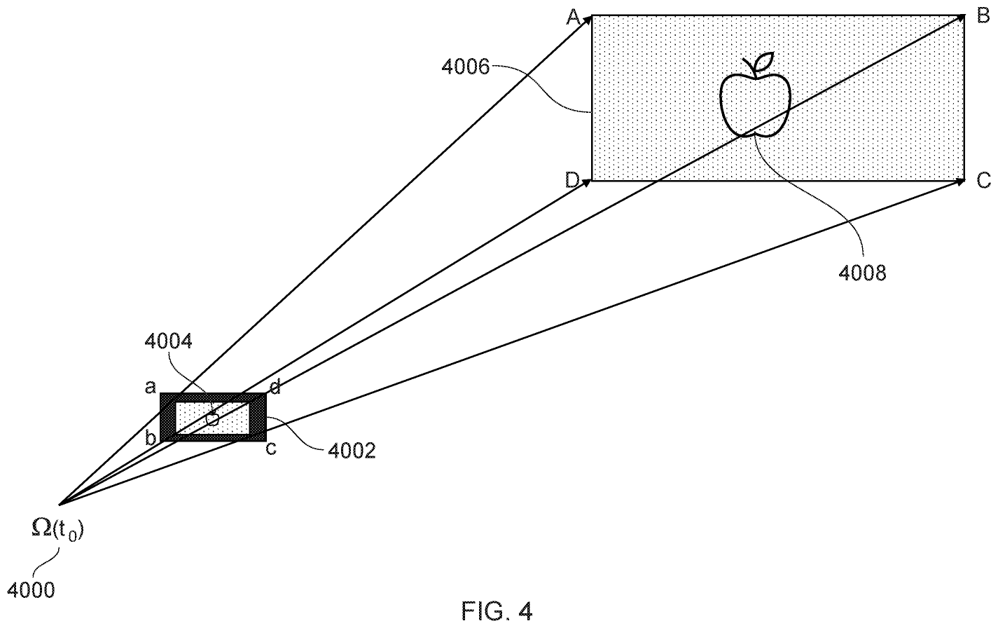

Further details regarding the apparatus 3000 will now be described with reference to FIGS. 4 to 8 of the present disclosure.

### <Display Circuitry>

As explained with reference to FIG. 3 of the present disclosure, an apparatus 3000 according to embodiments of the disclosure may include a display circuitry 3002 which is configured to display an image on a display device, the display device being a first distance from a user viewing the image on the display device.

In some examples, the display circuitry 3002 is therefore a display controller, which issues instructions which causes an image to be displayed on a display device. However, the display device itself need not be part of the apparatus 3000. Rather, the display device can be external to the apparatus 3000 (e.g. an external display or monitor, for example). This may be the case when the display device is implemented as a fixed display within a vehicle (such as a fixed display within a car), for example. However, in other examples, the display device may in fact be internal to apparatus 3000. This may be the case when apparatus 3000 is implemented as part of a personal electronic device of a user (e.g. a mobile telephone or a tablet computing device, for example).

Consider, again, the example situation which has been described with reference to FIG. 2 of the present disclosure, where a person is travelling in an autonomous or semi-autonomous (hereafter, self-driving) car.

In this example situation, the person is a passenger who wishes to watch a film on a display in the self-driving car. The display may be a screen which is built into the self-driving car. Accordingly, the person issues an instruction or command which tells apparatus 3000 they wish to view a film on the screen. Upon receiving such an instruction or command, the display circuitry 3002 may then instruct the screen which is built into the self-driving car to display the film which has been selected by the user. The user can then watch the film on the screen.

Of course, the type of image which the display circuitry can cause to be displayed on a display device is not particularly limited. That is, while a person may decide to watch a film (movie) as the image on the display screen, the image could, alternatively, include a static image (such as a photograph), a videogame, a television programme, animated content, a video call, live-streamed video content, a user interface, a navigation system, a webpage, or the like. Indeed, the image may also include images which are formed of or at least include a portion of text. For example, a person may choose to read a message on the display device or a portion of a digital book (e-book). An image in accordance with the present disclosure includes any type of visual content which can be displayed on a display device.

The display circuitry may update the image on the display device when a further instruction or command is received from the user. Alternatively, the display circuitry may update the image on the display device when there is a change in the digital content. For example, in the case displaying video content on the display device, the display circuitry may update the display for each frame of the video content.

The distance between the user and the display device is defined as the first distance in the present disclosure. In some examples, this may be the distance between the user's observation position and the display device. Consider the example situation of FIG. 2 of the present disclosure, where a person is watching an image on a display device in a self-driving car. Here, the display device may have a fixed position within the self-driving car. This may be a fixed position on the dash-board of the car or on the back of a seat (e.g. on the back of a headrest), or alongside windows, substantially perpendicular to the intended direction of travel, or as a head-up display in the front windshield of the car for example. The distance between the person and the display device would remain fixed in this situation, as the person will watch the fixed display device from a predetermined location within the self-driving car (e.g. from the car seat facing the display device). However, in other examples, the display device may not have a fixed location. This may be a situation where the display device is part of a portable electronic device (e.g. a mobile telephone device). In this situation, it can be determined that the person will watch the display at a fixed distance (e.g. at arm length of the person) and/or the distance between the person and the display device can be measured (e.g. from a captured image of the user and/or the display device).

It will be appreciated that this distance is often a distance which is quite close to the person who is viewing the display. Moreover, the relative motion between the person and the display device is very small. That is, in the example where a person is travelling in a vehicle, both the person and the display device are on-board the vehicle. Therefore, both the person and the display device travel along with the vehicle. Alternatively, if the person is watching a display device while walking (as illustrated in panels 2004 and 2006 of FIG. 2 of the present disclosure) then the display device travels along with the person since the person is holding (or wearing) the display device. Therefore, relative motion between the person and the display device remains small such that the first distance (i.e. the distance between the person and the display device) often remains the same during the journey. However, any change of the relative position of the person and the display device can be measured (e.g. from a captured image of the user and/or the display device). Therefore, changes in the first distance can be accounted for by apparatus 3000 and/or display circuitry 3002.

### <Generating Circuitry>

As explained with reference to FIG. 3 of the present disclosure, an apparatus 3000 according to embodiments of the disclosure may include a generating circuitry configured to generate a virtual screen corresponding to the display device by projecting a line from the user and the display device to a second location, the distance of the second location from the user being greater than the first distance.

The condition of motion sickness arises owing to a discrepancy or contradiction between the different signals a person receives from their senses. Most typically, this arises when there is conflict between a person's sense of balance from their inner ear and a person's vision from their eyes. If their inner ear detects motion yet the image seen by their eyes does not show any motion then motion sickness may be experienced. Watching an image on a display device may exacerbate motion sickness, since when focused on the display device a person watching that display device will not see motion through their environment (e.g. the movement of a vehicle in which they are travelling).

The inventors have realized that the sense of discomfort and motion sickness when watching a display device can be alleviated through dynamic compensation of the image location on the display screen in accordance with the movement of the person (user) and the display device. For example, when the user and the display device are on-board a vehicle, the sense of motion sickness can be alleviated or reduced through dynamic compensation of the movement of the vehicle. Dynamic compensation of the image location on the display screen in accordance with the movement of the vehicle in this situation reduces the mismatch between the user's senses because the image seen by the user then moves in accordance with the sense of motion which has been detected in accordance with the user's other senses. Therefore, the problem of alleviating motion sickness when watching an image on a display device can be addressed.

In order to provide this compensatory movement of the image location, the generating circuitry of the present disclosure first generates a virtual screen. The virtual screen emulates a situation where the area of interest being watched (i.e. the display device) is actually located at a much greater distance from the user than the display device. Moreover, the virtual screen has a fixed location in a frame of reference external to the vehicle, such that any relative motion between the vehicle and the virtual screen can be accounted for through a change of image location on the display device. Thus, the generating circuitry generates a virtual screen which has a fixed location in a frame of reference external to the car and which is located far away from the user. This reduces motion sickness risk and alleviates the suffering of the user.

Consider, now, the example shown in FIG. 4 of the present disclosure. Here, an example of generating a virtual screen in accordance with embodiments of the disclosure is illustrated. This may correspond to an example situation where a user is viewing a display device while travelling in a vehicle (such as a car, a train, a plane or a boat, for example).

A user 4000 and an in-vehicle display device 4002 are illustrated in this example. The locations of the user 4000 and the in-vehicle display device 4002 may be the location of the user and/or the in-vehicle display device 4002 at an initial time to (where to corresponds to a time when the user first starts to watch an image on the display device or a time when the user's journey in the vehicle begins, for example).

In this example, the in-vehicle display 4002 has a rectangular shape defined by the points a, b, c and d. However, the present disclosure is not particularly limited in this regard, and any shape or type of display can be used.

On this in-vehicle display device, an image 4004 is displayed. This is the image seen by the user 4000 at time to.

The location of the user and the display device at time to may be predetermined (e.g. known from the location of the user and the display device at the start of the journey) or it may be detected by the detected device using information from one or more sensing devices. Further information regarding detection of the position and movement of the user and display device will be provided with reference to the detecting circuitry 3006 of apparatus 3000.

In order to generate the virtual screen, the generating circuitry 3004 projects a line from the user and the display device 4002 to a second location, the distance of the second location from the user being greater than the first distance. In other words, the generating circuitry traces a line from the initial position of the user 4000 to obtain at the second distance rectangle A, B, C, D (defining the virtual screen) and corresponding homothetic replication of the image 4004, as shown on the in-vehicle display device, on the virtual screen 4006. Each point on the virtual screen 4008 corresponds to the point on the display device 4004 projected to the second distance. For example, a line from the initial position of the user 4000 to point a on the display device 4004 extended or projected to the second distance corresponds to the point A on the virtual screen 4006.

Of course, projecting or extending a line from the user and the display device to the second location will be understood to mean that the generating circuitry extrapolates the coordinates of the display device (relative to the user) to a second location in order to generate a virtual screen which is located at a greater distance from the user compared to the display device. In this way, a virtual screen at the second location can be generated by the generating circuitry.

In this specific example, the generating circuitry 3004 therefore projects a number of lines from the initial position of the user to the second location; that is in this example, the generating unit projects four lines (one from each of the four corners of the display device a, b, c and d) to the second location. However, the present disclosure is not particularly limited to this example. In other examples, the generating circuitry may project a single line from the user and the display device to the second location in order to generate the virtual screen. For example, the generating circuitry may project a single line from the center of the display device to the center of the virtual screen in order to generate the virtual screen at the second distance. Therefore, the number of lines used by the generating unit to generate the virtual screen at the second location may be much greater or much less than the four lines used as an example in FIG. 4 of the present disclosure and is not particularly limited to this example.

The second distance (i.e. the distance of the virtual screen 4006 from the user 4000) should be a distance which is much larger than the distance between the user 4000 and the display device 4004. However, the precise distance at which the virtual screen 4006 is located from the user 4000 is not particularly limited in accordance with the present disclosure, so long as it is further from the user 4000 than the display device 4002. In some examples, the virtual screen remains at a fixed second distance from the user 4000 even if the user travels towards the display screen. That is, in some examples, the virtual screen may have a fixed location in a frame of reference external to the vehicle in all dimensions other than the direction of travel, along which the virtual screen has a fixed location relative to the user. In other examples, the virtual screen has a location which is fixed in a frame of reference external to the vehicle and the second distance may be a distance such that movement of the vehicle towards the virtual screen (in the direction of travel) is negligible in comparison to the second distance. However, the present disclosure is not particularly limited to these specific example situations, and the second distance may be any distance which is much larger than the distance between the user 4000 and the display device 4004.

The manner by which the generating circuitry 3004 projects a line from the user and the display device to a second location is not particularly limited in accordance with embodiments of the disclosure. For example, the generating circuitry 3004 may perform an extrapolation of a line from the user and the display device to the second distance. However, other ways of projecting the line from the user and the display device to the second distance can be used as required.

That is, projecting the line (such as a line from the center of the display device) to the second location is understood as extending the line (e.g. extrapolating) the line from the display device to the second location. In order to project the line to the second location, the generating circuitry may therefore trace a line from the display device to the second location. However, the present disclosure is not particularly limited in this regard. Indeed, any suitable way of performing the calculation to project the line from the display device to the second location may be performed by the generating circuitry as required depending on the situation. In addition, the line will be understood to be a virtual line; it is not a physical line in space which would be visible to a user. Rather, the line is a virtual line used as a construct to enable the generating unit to generate the virtual screen at the second location.

Moreover, it will be appreciated that, in general, the virtual screen which has been generated remains at a fixed location within a frame of reference external to the vehicle. Therefore, even if the vehicle moves, the virtual screen remains in the location generated by projecting the lines from the initial location of the user 4000 to the display screen 4002, to the second distance.

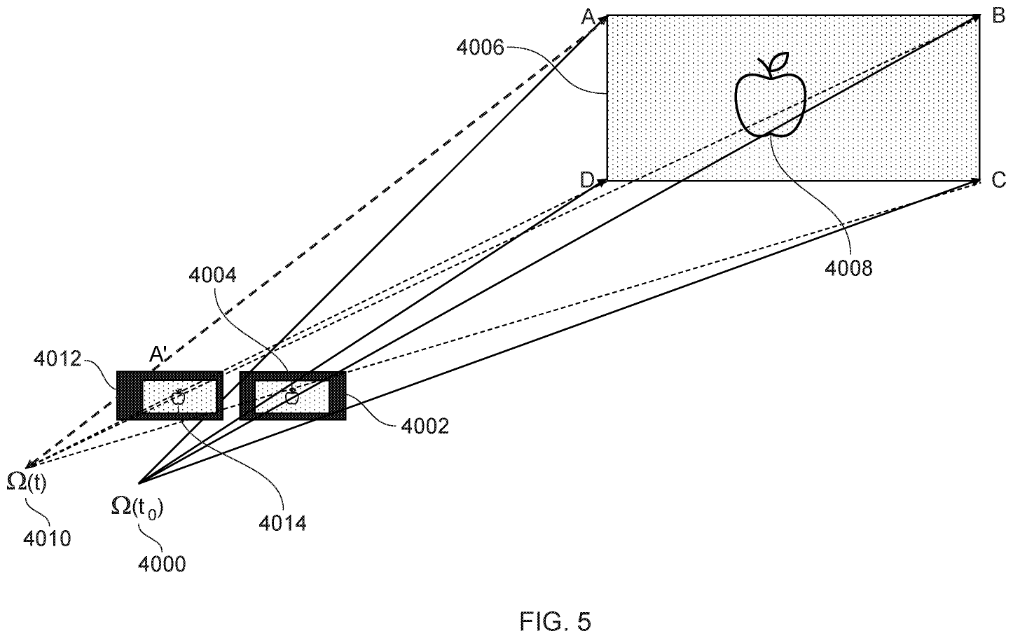

Notably, it will be appreciated that the user views the virtual screen 4006 on the display device 4002. That is, the user looks at the display device 4002 in order to see an image or representation of the virtual screen. The display device 4002 is thus a tool for viewing the virtual screen 4006. If the user looks out of the vehicle towards the location of the virtual screen without looking at the display device, then the virtual screen will not be seen. This is because the virtual screen is not a screen which exists in the physical world, but rather is a construct generated by the generating circuitry 3004 within a virtual environment which can be used in order to provide dynamic compensation for the movement of the vehicle and thus alleviate feelings of motion sickness. This will be explained with more detail with reference to FIG. 5 of the present disclosure.

In this way, the generating circuitry 3004 is able to emulate a situation where the area of interest being watched (i.e. the display device) is actually located at a much greater distance from the user than the display device and which remains in a fixed location in a frame of reference external to the vehicle.

### <Detecting Circuitry>

As explained with reference to FIG. 3 of the present disclosure, the apparatus 3000 comprises a detecting circuitry 3006 configured to detect a movement of the user and the display device to a new location relative to the virtual screen. By detecting the movement of the user and the display device relative to the virtual screen, it is possible to provide dynamic compensation for the movement which thus reduces the sense of motion sickness.

Consider, now, FIG. 5 of the present disclosure. FIG. 5 illustrates an example of determining a new image location in accordance with embodiments of the disclosure.

This example is the same as the example of FIG. 4 of the present disclosure. However, FIG. 5 shows the situation at a time after to, being a time when a movement of the user and the display device relative to the fixed virtual screen has been detected. That is, at time to the user is located at their initial position 4000. This may be the location of the user at the start of a journey, for example. Likewise, at this same time to the display device is located at its initial position 4002. As explained with reference to FIG. 4 of the present disclosure, generating circuitry 3004 generates the fixed virtual screen 4006 at this time (to) by projecting the lines from the user 4000 and the display device 4002 to a second distance.

However, at a later time t, the user and the display device have moved from their initial position. In the example of the user and the display device being located on-board a vehicle such as a car, this movement of the user and the display device from their initial position (i.e. their position at to) may be caused by the movement of the vehicle as it travels along its journey.

As such, at time t (being a time later than time to) the user is located at a new position 4010 and the display device is located at a new position 4012.

Notably, this movement of the user and the display device is a movement relative to the fixed virtual screen 4006. That is, while the user and the display device have moved with the movement of the vehicle, the virtual screen remains fixed at the same location 4006. This is because the generating circuitry 3004 generates the virtual screen based on the initial position of the user 4000 and the display device 4002 (i.e. their positions at to).

Of course, it will be appreciated that this relative movement of the user and the display device compared to the virtual screen can be either rotational and/or translational movement. Translational movement directly towards the screen, however, does not necessarily affect the display of the image on the display device in accordance with embodiments of the disclosure (as change of distance in comparison to the second distance may be very small).

The way in which the detecting circuitry 3006 detects the movement of the user and the display device from their initial positions (i.e. their positions at to) is not particularly limited in accordance with embodiments of the disclosure. Rather, this depends on the situation to which embodiments of the disclosure are applied.

Consider the example situation described with respect to panels 2004 and 2006 of FIG. 2 of the present disclosure. In this example, a person is travelling by walking along while watching an image on their portable electronic device. Here, the detecting circuitry 3006 may detect the movement of the user and the display device relative to the virtual screen based on information from one or more sensors on the user and/or within their portable electronic device. These sensors may include one or more of gyroscopic sensors, accelerometer sensors or the like. Alternatively, the detecting circuitry 3006 may use a Global Navigation Satellite System (GNSS), such as GPS signals from the user's device, in order to detect the movement of the user and the display device relative to the virtual screen.

It will be appreciated that in some examples, the sensors (such as the gyroscopic sensor and/or the accelerometer sensor) may be included as part of apparatus 3000. However, in other examples, apparatus 3000 may receive information from external sensors (e.g. sensors within the user's portable electronic device and/or wearable technology such as a smart-watch or the like).

In addition, the detecting circuitry 3006 may detect movement of the user and the display device based on an image of or captured by one or of the user and/or the display device. Movement of the user and/or the display device through an environment relative to the virtual screen can be determined from these captured images by the detecting circuitry 3006.

Consider, now, the example situation described with reference to panel 2006 of FIG. 2 of the present disclosure. In this example, a person is travelling in a self-driving car. During the journey, they are watching an image on a display device within the car. Here, the detecting circuitry 3006 of apparatus 3000 may detect the movement of the user and the display device relative to the virtual screen based on information from one or more sensors on the user and/or within the display device. However, alternatively or in addition, the detecting circuitry 3006 of apparatus 3000 may also detect the movement of the user and the display device relative to the virtual screen based on the movement of the car itself. That is, since the user and the display device are travelling in the car, they will move with the car as it progresses along its journey. Therefore, if the car moves relative to the virtual screen, the user and the display device will also move relative to the virtual screen. This provides a particularly efficient and reliable method of detecting the movement relative to the virtual screen as sensors contained within the car (or indeed any other type of vehicle in which the user is travelling) can be used for detection of movement. In this situation, the distance from the user to the display device remains the first distance when the user and the display device move to a new location relative to the virtual screen as the user and display device have not moved relative to each other (rather, the movement is dictated by the movement of the car).

When using the sensors contained within the car to detect the movement of the user and the display device, the detecting circuitry 3006 may determine the motion of the car using one or more of gyroscopic sensors, accelerometer sensors, GPS signals, mapping information and/or control signals for controlling the motion of the vehicle. The mapping information may include a map of the route that will be taken by the car during the journey. Correlating the GPS signals with the mapping information may enable the detecting circuitry to more accurately determine the motion of the vehicle relative to the virtual screen. Moreover, if the car is a self-driving car, control signals controlling the car may be used by the detecting circuitry 3006 in order to determine movement of the car. For example, if a control signal to control the car indicates an instruction for the car to turn left, then this information regarding the turn can be used by the detecting circuitry in order to detect the movement of the car.

Likewise, even if the car is not a self-driving car, then control signals can be used in order to determine the movement of the car. That is, a sensor which detects control signals such as a rotation of the steering wheel by the driver can be used by the detecting circuitry 3006 in order to determine the movement of the car. This enables the detecting circuitry to respond directly to the control of the car when detecting the movement.

Of course, it will be appreciated that the present disclosure is not particularly limited to the above-described example situations. That is, the detecting circuitry 3006 can use any suitable source or combination of sources of information in order to detect the movement of the user and the display device relative to the virtual screen depending on the situation to which the embodiments of the present disclosure are applied.

### <Determining Circuitry>

As explained with reference to FIG. 3 of the present disclosure, apparatus 3000 may further comprise a determining circuitry 3008 configured to determine a new image location by projecting a line from the virtual screen location to a new location of the user.

The determining circuitry 3008 of apparatus 3000 uses information obtained by the detecting circuitry 3006 regarding the location of the user and the display device in order to determine a dynamic adjustment to the image displayed on the display device to account for movement relative to the virtual screen.

Consider, again, the example situation illustrated with respect to FIG. 5 of the present disclosure. Here, an example of determining a new image location in accordance with embodiments of the disclosure is illustrated.

In this example, the user and display device are originally located at positions 4000 and 4002 (this is at time to). The virtual screen generated by the generating circuitry 3004 is then located at 4006. At a time, which is a time after to, the user and display device have moved to a new location of 4010 and 4012 respectively. The movement of the user and the display device to this new location may be detected by the detecting circuitry 3006.

In order to determine a new image location, the determining circuitry 3008 may perform a back-projection from the virtual screen 4006 to the new location of the user. This new image location emulates how the user's view of the virtual screen will have changed based on the movement from 4000 to 4010.

Accordingly, in this example, the determining circuitry 3008 may project a line from each location on the virtual screen to the location of the user. This mapping enables the determining circuitry 3008 to determine where each part of the image displayed on the virtual screen should be rendered or displayed by the display device in order to account for the movement which has occurred relative to the virtual screen. For example, point A of the virtual screen (being the top left-hand corner of the virtual screen) has a location which has been generated by projecting a line from the initial location of the user and the display screen to a second distance. In order to determine where this point on the virtual screen should be displayed (i.e. the new image location) the determining circuitry 3008 then projects a line from the location of the point A on the virtual screen to the new location of the user 4010 at time t. The new image location A′ then falls on this line at the point of intersection of the display screen 4012.

Each point on the virtual screen 4006 can be mapped to the new location of the display screen 4012 in this manner. As such, a new image location 4014 can be determined by the determining circuitry 3008. This new image location emulates the user's view of the virtual screen from the new location which has been detected by the detecting circuitry 3006 such that dynamic compensation for the movement of the movement is provided.

The manner by which the determining circuitry 3008 performs the calculation to project the points from the virtual screen to the new location of the user is not particularly limited in accordance with embodiments of the disclosure. Any suitable method which can be used to form a line connecting a point on the virtual screen to the new location of the user can be used as appropriate.

Moreover, it will be appreciated that the determining circuitry 3008 may determine a new image location every time that the detecting circuitry 3006 detects that a change of position of the user and the display screen has occurred. Alternatively, the determining circuitry 3008 can determine a new image location on a periodic basis. If a periodic basis is used, then the determining circuitry 3008 may determine a new image location a number of times per second, for example. However, the periodic basis is not particularly limited in this regard, and the new image location could be determined on a much longer or much shorter period than this.

Once the new image location has been determined, the determining circuitry 3008 of apparatus 3000 may provide information to the display circuitry 3002 to cause the display circuitry to display the image at the new image location. That is, at this stage, the display circuitry 3002 may be configured to display an image on the display device at the new image location for at least a portion of the image for which the new image location intersects with the display device.

In this manner, the display circuitry 3002 causes the display device to display the image at the new image location. In the case that the user is viewing an image such as a photograph on the display screen, the image will be the same image that was originally displayed on the display device when the display device was at the initial location 4002 (that is, image 4004, 4008 and 4014 are the same image in this example). However, if the user is viewing a video as the image on the display device, then the image displayed at the new image location may be a different video frame than the video frame which was shown on the display device when the display device was at the original location. As such, the image which is shown at the new image location may or may not be the same image as was shown on the display device when the display device was at the original location 4002 depending on the type of content being watched by the user.

By displaying the image at the new image location, the image which is seen by the user on the display device will dynamically adjust its location in accordance with the movement of the user and the display screen relative to the virtual screen. Accordingly, the sense of motion experienced by the user on the basis of information from senses such as their inner ear will be reflected in a corresponding movement in the image which is seen by the user on the display screen. This means that conflict between the user's different senses is reduced which thus alleviates motion sickness.

Of course, it will be appreciated that the display circuitry 3002 can only cause the display device to display the image at the new image location for the portion of the image where the new image location intersects the actual physical location of the display device. Therefore, if the new location of the user and the display device is very far removed the initial location of the user and the display device, then it may be the case that a significant portion of the image cannot be shown (i.e. that there is only a small overlap between the new image location and the actual physical location of the display device). Therefore, in the case of very significant movement of the user and the display device from their initial location, it may become difficult for the user to view content on the display device. Moreover, when the movement from the initial location is very large, the feeling of motion sickness experienced by the user may be stronger.

As such, in some examples, apparatus 3000 may be configured to adapt the display of the image on the display device when the movement of the user and the display device relative to the virtual screen is above a threshold value, enabling significant movement from the initial location to be efficiently accounted for.

The threshold value which is used to trigger the adaptation of the display of the image is not particularly limited. This threshold value may be a predetermined threshold value. For example, it may be a value which is set by the user in advance depending on their individual preferences.

Alternatively, it may be determined based on the context of the situation (including, for example, the type of vehicle in which the user is travelling). In some examples, the threshold value may correspond to a degree of movement for which no portion of the new image location intersects with the display device. In this case, if the movement of the user and display device was such that there was no overlap between the actual new location of the display device and the new image location, the apparatus 3000 would adapt the image for display. Such an adaptation may include adjusting the new image location such that at least a portion (e.g. 50%) of the new image location intersects with the display. If the new image location is adjusted in this manner, a visual indicator may be provided on the screen in order to inform the user that the maximum movement of the image has been reached. However, the present disclosure is not limited to this regard.

Alternatively, in some examples, the threshold value may be determined based on an amount of movement within a predetermined time (i.e. when the rate of movement is above a threshold value). That is, if the position of the user and the display device varies rapidly with time or follows a particular pattern of motion, then adaption of the display may be triggered. This may correspond to significant turbulence when viewing an image on a display device while travelling in a plane, for example. Alternatively, this may correspond to a particularly bumpy or winding portion of the road when travelling in a car.

In some examples, adapting the display of the image on the display device may include displaying a predetermined image to replace the image on the display device. If the movement of the user and the display device is above the threshold value, then the user may be more likely to experience motion sickness when viewing the display, even if the location of the image is dynamically adjusted. Accordingly, a predetermined image may be shown in this case to further reduce the user's sense of motion sickness. This may include a blank image (such that the user stops looking at the display) or an image with a predetermined property. The image with a predetermined property may be an image that instructs the user to stop looking at the display (i.e. an image with a visual warning that the movement is very large) and/or an image with a property that is known to reduce the feeling of motion sickness. That is, for example, if the user is watching video content such as an action movie then the user may be more susceptible to motion sickness (as the conflict between the movement of the objects within the image content with the sense of motion of the user may be increased). Accordingly, in this case, the display may replace the image with an image which is likely to reduce the user's sense of motion sickness. This could be a relaxing photograph for example. Alternatively, it could be a relaxing image which has been chosen in advance by the user.

However, even if the image which is displayed to the user is adapted by the apparatus when the movement is above the threshold value, apparatus 3000 may still generate audio associated with the image when the display of the image on the display device has been adapted. That is, if the user is watching video content on the display, then the audio associated with that video can still be generated even if the display is adapted (e.g. replaced with a blank screen or a warning to stop watching the video content). As such, even if the user is unable to watch the display owing to a feeling of motion sickness, they can still listen to the audio associated with that content. Therefore, the user can continue to enjoy the content even in the event of significant movement from the initial location. Moreover, if the user is unable to watch the display (owing to a feeling of motion sickness) then the audio may be adapted in order to provide the user with a verbal description of the video content. As such, the user can continue to enjoy the content even if they are unable to watch the display device.

Furthermore, in some examples, when there is audio associated with the image displayed on the display device, apparatus 3000 may be further configured to generate audio associated with the image when the image is displayed on the display device; and move a source location of the audio in accordance with the new image location when displaying the image on the display device at the new image location. That is, the origin of the sound being produced may be moved in accordance with the move of the image on the display device (i.e. the new image location which is determined by the determining circuitry 3008). In some examples, this may be performed apparatus 3000 controlling an audio production device (such as a speaker or the like) in a manner to control the virtual sound localization. The present disclosure is not particularly limited to this specific manner of moving the location of the audio in accordance with the new image location which has been determined. However, it will be appreciated that moving the origin of the audio being produced in accordance with the new image location which has been determined may further reduce the conflict between the different senses of the user and the motion of the vehicle and thus may further alleviate the sense of motion sickness experienced when watching an image on the display device.

In some examples, a passenger may be watching A/V content on a display device in a vehicle. When watching A/V content in a moving vehicle, the passenger may experience a sense of motion sickness as a sense of motion detected by the passenger may not match the motion seen by the passenger using their eyes when watching the display device. Therefore, in some examples, the A/V content being watched by the passenger may be layered over a live video representation of the horizon obtained from a video camera (or other type of image capture device). Such a video camera (or other image capture device) may be arranged either on the inside or outside the vehicle. However, in either case, it will be appreciated that the video camera will be attached to the vehicle such that it moves with the vehicle. Therefore, the live video representation of the horizon captured by the video camera will change as the vehicle moves. Accordingly, the passenger may then still see the movement of the vehicle (relative to the horizon) as they are watching the A/V content on a display device in the vehicle. This may further reduce the sense of motion sickness experienced by a user. Of course, it will be appreciated that the determining circuitry 3008 of apparatus 3000 can then determine a new image location at which the display circuitry 3002 should display the image which is layered over the live video representation of the horizon based on the movement of the passenger and the display device relative to the virtual screen (located at the horizon, for example). Thus, the A/V content which is layered over the live video representation of the horizon captured by the video camera will move to a new display location based on the movement of the vehicle and the live video representation of the horizon captured by the video camera will change as the vehicle moves along the road. Therefore, the passenger watching the display device will see the motion of the vehicle fully reflected on the display device and a sense of motion sickness will be further reduced.

In addition, apparatus 3000 may be configured to reduce the contrast of the live video representation of the horizon with respect to the A/V content layered over this live video representation of the horizon. Such a change of contrast may change the attention of the user such that they focus either on the A/V content or the live video representation of the horizon. Such a change in contrast may be performed by apparatus 3000 depending on the level of motion sickness being experienced by the user (either determined based on an observation of the user or, alternatively, based on a prediction of the level of motion sickness in accordance with conditions of the journey). If the user is experiencing a low level of motion sickness, the contrast of the live video representation of the horizon with respect to the A/V content may be reduced, such that the live video representation of the horizon becomes less noticeable to the user. This means that the user will focus mainly on the A/V content which is displayed by apparatus 3000. However, if the user is experiencing an increased level of motion sickness (despite the adaptive movement of the image location of the A/V content by apparatus 3000) then apparatus 3000 may increase the contrast of the live video representation of the horizon with respect to the A/V content, such that the live video representation of the horizon becomes more noticeable to the user. By focusing on the live video representation of the horizon, the sense of motion sickness of the user may further be reduced. Therefore, by adapting the contrast of the live video representation of the horizon shown on the display to the contrast of the A/V content layered over this live video representation, based on the sense of motion sickness of the user, it is possible to further reduce the level of motion sickness of the user.

Furthermore, in some examples, the level of brightness outside the vehicle may change as the vehicle travels along its journey. For example, the vehicle may begin travelling during the day when it is bright outside. Then, it may become darker outside the vehicle if the vehicle is still travelling along the journey later in the day (e.g. in the evening or at night). Alternatively, it may become darker outside the vehicle if the weather changes (e.g. if it becomes very cloudy, for example). If a passenger is watching a display device in the vehicle then they may become accustomed to the brightness of the screen. As such, if the passenger has to become the active driver (e.g. if the passenger needs to take over control of the vehicle) then they may be unable to see outside the vehicle properly if it has become dark outside and they are accustomed to the brightness of the screen. This is because it takes a period of time for a person's vision to adjust to a lower level of brightness. Accordingly, in some examples, apparatus 3000 may monitor the brightness outside the vehicle using the video camera which captures a live video representation of the horizon (or any other suitable device to monitor the brightness outside the vehicle). Then, apparatus 3000 may adapt the brightness of the display device in accordance with the level of brightness outside the vehicle. If it becomes darker outside, apparatus 3000 may reduce the brightness of the display device, for example. Accordingly, if the passenger has to take control of the vehicle their vision will be more appropriately accustomed to the level of brightness outside the vehicle. This may further improve safety when the passenger is travelling in watching a display device in a vehicle for which they may have to become the active driver.

In this manner, the determining circuitry 3008 of apparatus 3000 can determine a new image location at which the display circuitry 3002 should display the image based on the movement of the user and the display device relative to the virtual screen. Accordingly, a user's sense of motion sickness when watching an image on a display device can be alleviated since the display of the image on the display screen is adjusted in accordance with the movement of the user and the display device.

### <Example Situations>

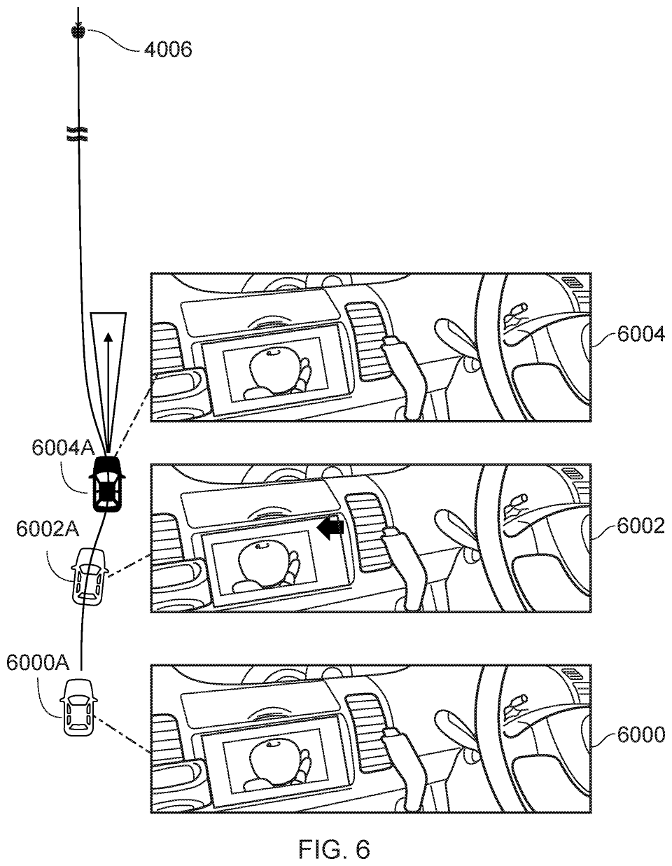

An example of the application of the apparatus 3000 of the present disclosure to an example situation in accordance with embodiments of the disclosure will now be described with reference to FIGS. 6, 7 and 8 of the present disclosure.

FIG. 6 illustrates an example situation to which embodiments of the disclosure are applied.

In this example, a user is watching a video while travelling in a car. The car may, optionally, be a self-driving car (e.g. an autonomous or semi-autonomous vehicle). Alternatively, the car may be a car which is manually driven by a driver. In this case, the user may be a passenger who is being driven in the car by the driver.

The user begins the journey in the car travelling along a road at location 6000A. At this time, the user may request that an image is shown on a display within the vehicle 6000. That is, the initial location of the user and the display device within the vehicle may be set at the time corresponding to the location of the car at 6000A. As such, the image is centered on the display within the vehicle 6000 and the generating circuitry 3004 of apparatus 3000 generates a virtual screen location 4006. The user is thus viewing the virtual screen 4006 through the display within the vehicle 6000.

As the car is driving directly towards the virtual screen location, there is no relative motion between the user, the display device and the virtual screen such that the image displayed on the display device remains centered on the display device.

Then, after some time has passed, the car may reach a gentle turn in the road at location 6002A. Accordingly, the car (under the control of the driver or as part of the autonomous driving of the vehicle) may move relative to the virtual screen. This is illustrated in FIG. 6 at location 6002A as the car begins to turn to the right.

At this stage, the user and the display device move relative to the virtual screen. The user's view of the virtual screen through the display device will thus have changed. Accordingly, the detecting circuitry 3006 of apparatus 3000 detects this movement. The determining circuitry 3008 of apparatus 3000 determines a new image location of the image on the display and causes the display circuitry 3002 of apparatus 3000 to display the image at this new location. Accordingly, as the car turns to the right, the image seen by the user on the display device in the car 6002 (corresponding to the location of the car at 6002A) moves to the left. Thus, as the user senses and experiences the movement of the car to the right as the car turns around the gentle corner, the user can see this movement reflected in the location of the image displayed on the display device since the image on the display device (representing a view of the fixed virtual screen location through the display device) moves accordingly. Conflict between the user's senses is therefore reduced and the feeling of motion sickness experienced by the user when watching an image on the display device can be alleviated.

Then, at location 4006, the car straightens out and completes the gentle turn to the left such that it is again facing directly towards the virtual screen. Accordingly, apparatus 3000 updates the image which is displayed on the display device such that the image is centered on the display device. The user can therefore continue to enjoy watching the image on the display device as the car travels along the road.

Of course, it will be appreciated that if the car, instead, turns to the left, the image on the display may move towards the right as the new image location (representing a view of the virtual screen through the display device) will be on the right hand side of the display. Thus, any movement of the user and the display device as the car travels along the road relative to the fixed virtual screen location can be reflected through a corresponding movement of the image on the display.

While the image location has only been illustrated in FIG. 6 of the present disclosure for three locations of the car relative to the virtual screen (i.e. for locations 6000A, 6002A and 6004A), it will be appreciated that the image location may be updated on the display continuously by apparatus 3000 as the car travels along the road. That is, the user will not experience the image on the display jumping from one location to the other. Rather, the display circuitry 3002 may cause the image to smoothly slide from one image location in substantially real-time with the movement of the car relative to the virtual screen. This is possible because of the efficient manner by which the new image location is determined by apparatus 3000 in accordance with the movement of the user and the display relative to the virtual screen.

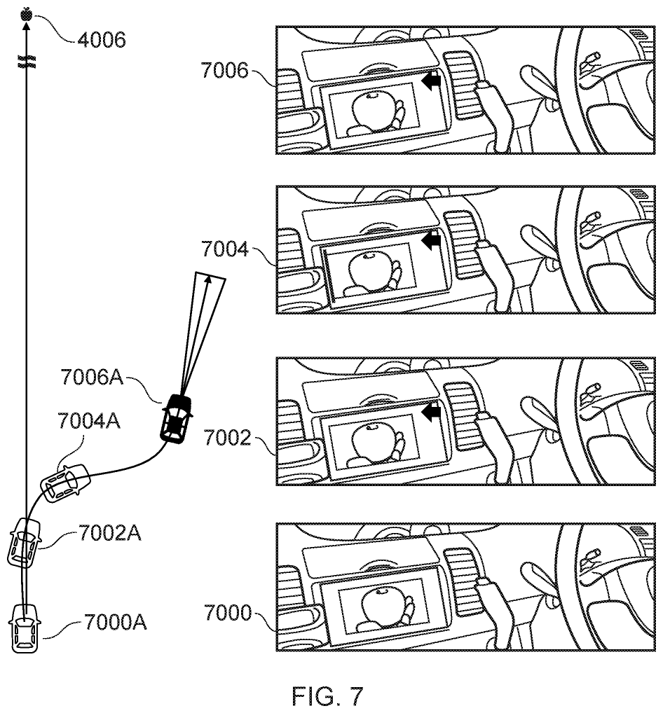

Referring now to FIG. 7 of the present disclosure, an example situation to which embodiments of the disclosure can be applied is illustrated. In this example a user is watching a video while travelling in a car, similar to the situation described with reference to FIG. 6 of the present disclosure. However, in this example, the car travels along a road and experiences a very sharp change in direction (e.g. follows a very sharp corner).

The journey begins at location 7000A and an image is displayed to a user on a display device in the car 7000. As this is the beginning of the journey (i.e. the initial location of the user and the display device) the virtual screen is generated at location 4006 (i.e. directly ahead of the car at location 7000A) and the image on the display device 7000 is, accordingly, centered on the display device.

As the car travels along the road, it reaches the corner at location 7002A and begins to turn (change direction) such that it follows the road round the corner. There is thus a relative motion between the user and the display device on-board the car and the virtual screen which has been generated at location 4006. Following a process similar to that described with reference to FIG. 6 of the disclosure, apparatus 3000 updates the display such that the image shown on the display moves towards the left. As such, the user can see a visual representation of the sense of motion experienced as the car turns the corner and the sense of motion sickness is reduced when watching the image on the display device.

However, as the car continues to turn round the corner in the road, the relative motion between the user, the display device and the virtual screen becomes very large (location 7004A in FIG. 7). As such, the new location of the image (determined by the determining circuitry 3008 of apparatus 3000) may be above a threshold value from the display device. That is, the movement of the image on the display device may be such that a significant portion of the image would not be visible on the display device (e.g. the intersection between the display device and the new image location would be very small). As such, in this example, apparatus 3000 may adjust the new image location once the threshold has been reached such that a predetermined portion of the image remains displayed on the display device (e.g. 75% of the image, for example). Therefore, as the car turns round the corner, the image shown on the display is adapted to represent the movement of the car relative to the virtual screen. However, once the amount of motion exceeds the threshold, the movement of the image on the display is adjusted such that a predetermined portion of the image remains on the display. Therefore, even when the movement relative to the virtual screen is large, the user is able to enjoy watching the image on the display. However, in this example, a warning indicator is provided (the red line on the display in the car 7004). The user can therefore understand from this warning that the risk of motion sickness is increased, owing to the significant movement of the car relative to the virtual screen. The user may therefore decide not to continue watching the image on the display device at this time.

As the car travels along the road, it completes the turn to the right and begins to turn once again to the left (i.e. towards the virtual screen). At location 7006A, the relative movement of the user and the display device in the car from the virtual screen becomes less than the predetermined threshold value. As such, the adaptation of the display performed by apparatus 3000 stops such that the new image location once again reflects the movement relative to the virtual screen. Accordingly, while the car begins to turn back towards the virtual screen, the image on the display device will move back towards being centrally located on the display device.

In the example of FIG. 7, a user is therefore able to enjoy watching an image on a display device with a reduced sense of motion sickness even when the amount of movement is very large.

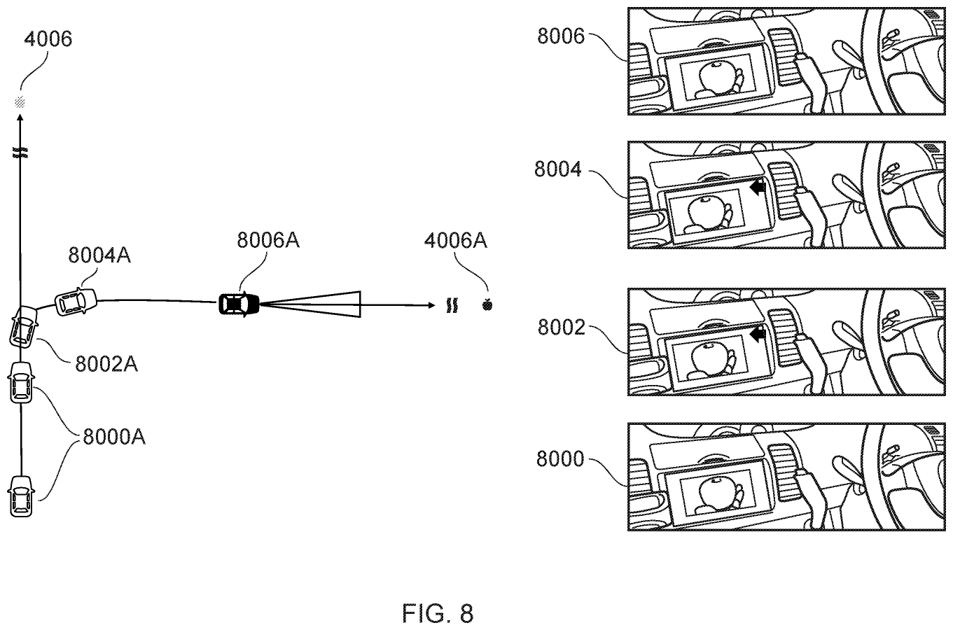

Referring now to FIG. 8 of the present disclosure, a further example situation to which embodiments of the disclosure can be applied is illustrated. In this example, a car travels along a road and turns sharply to the right, before continuing along a new road running perpendicular to the original road.

The journey begins at location 8000A. This may correspond to the time at which a user travelling in the car requests that an image is shown on the display device in the car. Accordingly, at this stage (i.e. when the car is located at 8000A) the generating circuitry 3006 of apparatus 3000 generates the virtual screen at the location 4006 and the display circuitry 3002 displays the image on the display device in the car 8000 such that the image is centrally displayed on the display device (i.e. representing a view looking directly at the virtual screen 4006 which has been generated).

As the car travels along the road, it travels directly towards the virtual screen 4006. Accordingly, the image shown on the display device in the car remains centered on the display device.

Then, at location 8002A, the car begins a turn towards the right. This may correspond to the driver of the car turning to join a new road. Since there is relative motion between the virtual screen 4006 which has been generated (based on the location of the car 8000A, and thus the location of the user and the display device, at the start of the journey) the determining circuitry 3008 of apparatus 3000 will determine that the display circuitry 3002 should display the image on the display device at a new image location. Therefore, as the car turns towards the right at location 8002A, the image on the display device in the car 8002 dynamically moves towards the left to reflect the change of view of the virtual screen as observed through the display device.

As the car continues to turn onto the new road, the movement or distance between the direction the car is facing and the virtual screen becomes very large. As such, the predetermined threshold may be reached (if such a predetermined threshold is imposed) and the image location on the display will reach its maximum displacement from the center of the screen. Even if the car continues round the corner, no further movement of the image on the display screen will occur. Therefore, if the car continues to travel along the new road (in the new direction) then the original location of the virtual screen which has been generated may no longer be appropriate to ensure that the sense of motion sickness experienced by a user when watching an image on a display is reduced.

Therefore, in some examples of the present disclosure, the apparatus 3000 may be configured to determine whether the movement or distance between the direction the car (and thus the user and the display device) is facing and the virtual screen remains above the predetermined threshold for a predetermined period of time. When the movement of the user and the display device relative to the virtual screen remains above the threshold value for a predetermined amount of time, apparatus 3000 may be configured to move the virtual screen to a new virtual screen location corresponding to the location defined by projecting a line from the new location of the user and the display device to a third location, the distance of the third location being the same as the second location.

As such, in this example, when the car has been travelling along the new road for a predetermined period of time and reaches the new location 8006A, apparatus 3000 determines that the virtual screen location should be moved (i.e. a new virtual screen should be generated) which is centered on the new direction of travel of the car. Therefore, at this time, the virtual screen 4006 is moved to a new virtual screen location 4006A corresponding to a location at the second distance determined by projecting a line from the new location of the user and the display device (i.e. their location at 8006A) to this second distance. In other words, the virtual screen location is reset so that a new initial location of the virtual screen is defined corresponding to the location of that virtual screen when it is based on the location of the car (and thus the user and display device) at location 8006A.

Once the virtual screen location has been moved to the new virtual screen location, the user and the display device will be travelling directly towards this new virtual screen location as illustrated in FIG. 8 of the present disclosure. Accordingly, at this time, the determining circuitry 3008 of apparatus 3000 will determine that the display circuitry 3002 should control the display device such that the image is displayed to the user centered from the display screen of the car 8006.

Any further change in direction of the car relative to this new virtual screen location will be reflected in the location of the image on the display in the same manner as described as for the original virtual screen location. A sense of motion sickness when watching an image on a display when travelling along this new road can therefore be alleviated.

It will be appreciated that the predetermined period of time which is used in order to determine whether or not to move the virtual screen to a new location is not particularly limited in accordance with the present disclosure. Indeed, the length of time which is set as the predetermined period of time may depend on the situation to which embodiments of the disclosure are applied. This may depend on a user preference, the context of the situation (e.g. the type of vehicle and the speed at which the vehicle is travelling), one or more additional sources of information or the like. An example of the one or more sources of information may include mapping information. If this mapping information indicates that the car has turned on to a new road and will remain travelling in this new direction, then the predetermined time period for moving the location of the virtual screen may be reduced. Alternatively, in some examples, if the mapping information shows that a series of significant changes in direction will occur, the predetermined time period may be increased such that a degree of hysteresis to the changes of direction is provided. This prevents sudden changes in the location of the image shown on the display and thus improves the viewing experience of the user while alleviating the feeling of motion sickness.

In some examples, once the predetermined period of time has expired and the movement remains above the predetermined threshold, apparatus 3000 may be configured to incrementally moving the virtual screen to the new virtual screen location over a second predetermined period of time. That is, the virtual screen may be gradually moved from the original location 4006 to 4006A by apparatus 3000 as the car continues to travel along the new road. This means that the image shown on the display device to the user will gradually move back towards the center of the screen as the virtual screen moves to its new location 4006A centered in front of the car. Accordingly, even when the virtual screen moves to its new location in front of the car, a sense of discomfort for the user viewing the image on the display can be avoided.

If the user travels along the new road for some time and then changes direction of travel to a new direction, then the virtual screen may, again, be moved to a new location as required.

Of course, it will be appreciated that while an application of the apparatus 3000 to a number of example situations has been provided with reference to FIGS. 6 to 8 of the present disclosure, the embodiments of the present disclosure are not particularly limited in this regard. That is, the embodiments of the present disclosure can be used in order to reduce or alleviate the sense of motion sickness of a user when viewing a display screen in many other types of situations. These may include situations where the car shown in these examples follows a road very different from the example roads which have been shown. Moreover, these may include situations where a vehicle other than a car is used. In fact, the embodiments of the disclosure be applied to situations where the display screen is not onboard a vehicle at all, but rather moves owing to the movement of a person walking along carrying the display device (such as the example described with reference to panels 2004 and 2006 of FIG. 2 of the present disclosure).

### <Advantageous Technical Effect>

The apparatus 3000 provided in accordance with embodiments of the disclosure (as described with reference to FIGS. 3 to 8 of the present disclosure) is configured to provide a dynamic adaptation of the image displayed on a display device in accordance with a detected movement of the user and the display device. This reduces, or alleviates, the sense of motion sickness experienced by a user when watching an image on the display device as the user and the display device move from one location to another.

Of course, it will be appreciated that advantageous technical effects provided by embodiments of the disclosure are not particularly limited in this regard. Other advantageous technical effects will become apparent to the skilled person when reading the disclosure.

### <Method>

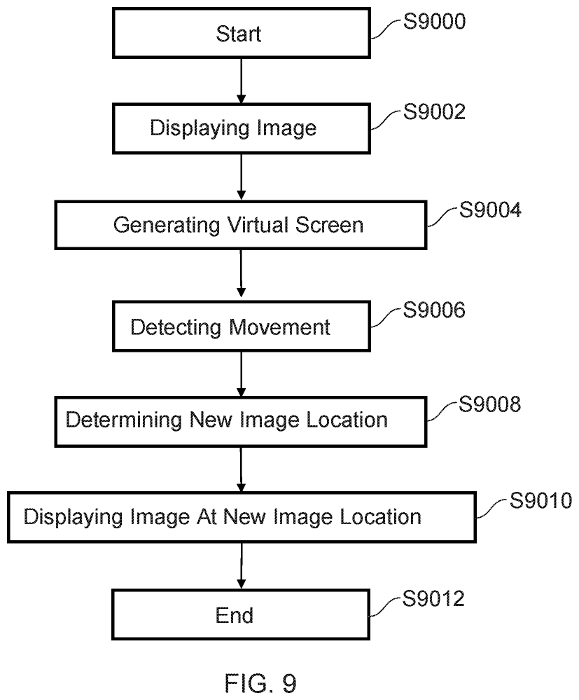

Turning now to FIG. 9 of the present disclosure, a method of alleviating motion sickness in a user viewing an image on a display is illustrated. This method may be performed by an apparatus such as apparatus 1000 of the present disclosure, for example. Furthermore, a computer program product comprising instructions which, when implemented by a computer, cause the computer to perform the method illustrated in FIG. 9 of the present disclosure may be provided.

The method illustrated in FIG. 9 starts at step S9000 and proceeds to step S9002. The start of the method may, in some examples, be a time at which an instruction to display an image on a display device is received. However, the present disclosure is not particularly limited in this regard and the start of the method may correspond to any other time (including, for example, the start of a journey in a vehicle).

In step S9002, the method comprises displaying an image on a display device, the display device being a first distance from a user viewing the image on the display device. The image may include a video being watched by a user, for example. Once the image has been displayed, the method proceeds to step S9004.

In step S9004, the method comprises generating a virtual screen corresponding to the display device by projecting a line from the user and the display device to a second location, the distance of the second location from the user being greater than the first distance. This virtual screen emulates a display device being watched by a user which is fixed and far away relative to the user and the display device.

The method then proceeds to step S9006.

In step S9006, the method comprises detecting a movement of the user and the display device to a new location relative to the virtual screen. The way in which this movement is detected is not particularly limited. However, in a case where the user is travelling in a vehicle, said movement may be detected by the corresponding movement of the vehicle, for example.

The method then proceeds to step S9008.

In step S9008, the method comprises determining a new image location by projecting a line from the virtual screen location to the new location of the user. By back-projecting the new image location from the virtual screen location in this manner, the movement of the user and the display device relative to the virtual screen can be accounted for. Then, the method proceeds to step S9010.

In step S9010, the method comprises displaying an image on the display device at the new image location for at least a portion of the image for which the new image location intersects with the display device. The user can then see movement of the image which reflects the actual movement of the user and the display device. A sense of conflict between the user's senses can therefore be alleviated which reduces the motion sickness risk.

The method then proceeds to and ends with step S9012. In some examples, the method may proceed to step S9012 when the user stops watching an image on the display device. Alternatively, in some examples, the method may proceed to step S9012 at the end of a journey. However, the present disclosure is not particularly limited in this respect.

Of course, it will be appreciated that the method of the present disclosure is not limited to the specific arrangement of the method steps which are illustrated in the example of FIG. 9 of the present disclosure. Other method steps may be included in this method as appropriate. Moreover, these method steps do not have to be performed in the order illustrated in FIG. 9; a number of the method steps may be repeatedly performed and/or performed in parallel with respect to one or more of the other method steps illustrated in FIG. 9.

### Second Embodiment—Planning Content

As described with reference to FIG. 2 of the present disclosure (and also in the Background) there are a wide range of situations where a person viewing content on a display, particularly when travelling (i.e. moving from one location to another), may experience motion sickness. Therefore, it is desired for at least these reasons to provide an apparatus, method and computer program product which can alleviate motion sickness experienced by a user.

In the first embodiment of the disclosure, an apparatus, method and computer program product are provided which can perform dynamic compensation for the movement in order to adjust an image displayed on a display device. Therefore, the first embodiment of the disclosure provides a way of adapting the image which is displayed in order to compensate for movement as it occurs (i.e. in a substantially real time environment). In other words, apparatus 3000 of the first embodiment of the disclosure responds to movement as it is detected in order to reduce motion sickness of the user.

In this second embodiment of the disclosure, an apparatus, method and computer program product for selecting content for display during a journey to alleviate motion sickness are provided. This second embodiment of the disclosure therefore enables planning of content to select for an upcoming journey such that suitable content can be selected in advance which will reduce motion sickness experienced by a user in the portions of the journey which are most likely to cause motion sickness.

Advantageously, planning content for display in this manner enables appropriate content which can reduce the feeling of motion sickness in the user to be determined and prepared in advance which ensures that such content is available for display at the correct time in the journey. This ensures that the feeling of motion sickness in a user can be reduced or alleviated when watching content on a display.

### <Apparatus>

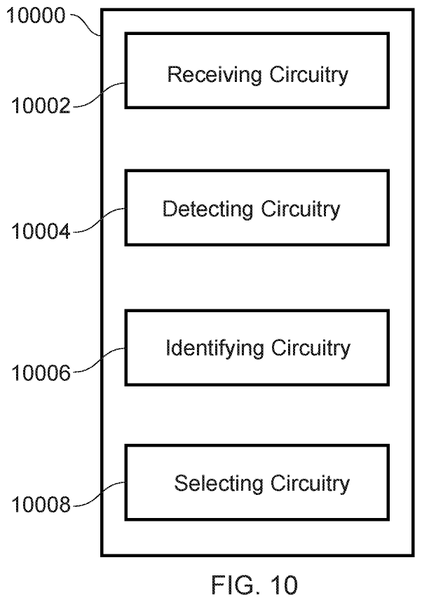

FIG. 10 illustrates an example apparatus in accordance with embodiments of the disclosure. The apparatus 10000 may be used in order to select content for a display during a journey to alleviate motion sickness.

The apparatus 10000 comprises a receiving circuitry 10002, a detecting circuitry 10004, an identifying circuitry 10006 and a selecting circuitry 10008.

The receiving circuitry 10002 may be implemented as processing circuitry configured to receive information indicative of a route for a journey.

The detecting circuitry 10004 may be configured to detect at least a first portion of the journey having a first predetermined characteristic. The detecting circuitry may detect the first portion of the journey having the first predetermined characteristic using the information indicative of the route of the journey received by the receiving circuitry. Alternatively, or in addition, the detecting circuitry may be configured to detect the first portion of the journey having the first predetermined characteristic using the information indicative of the route of the journey and one or more additional sources of information (e.g. mapping data indicating bends, predicted changes in altitude or the like). In this manner, the detecting circuitry can detect a first portion of the journey having the first predetermined characteristic.

The identifying circuitry 10006 may be configured to identify a time period corresponding to the first portion of the journey.

Finally, the selecting circuitry 10008 may be configured to select one or more items of content having a second predetermined characteristic for display over the time period.

In this manner, apparatus 10000 can identify a portion of the journey which, for example, is a portion most likely to cause motion sickness in the user, and may select content for that portion of the journey having a characteristic which, for example, will reduce or alleviate the feeling of motion sickness.

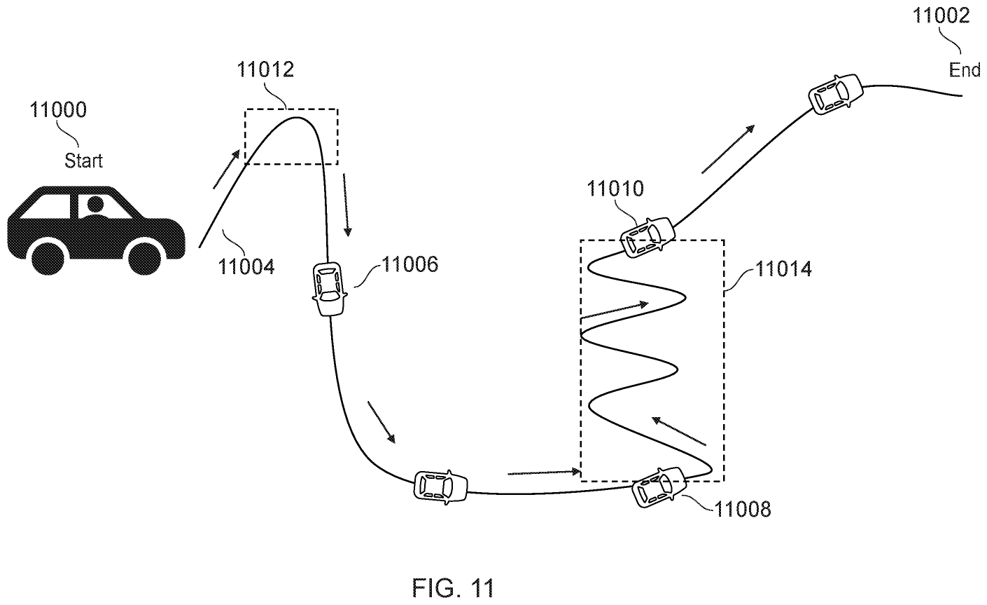

Further details regarding the apparatus 10000 will now be described with reference to FIGS. 11 to 14 of the present disclosure.

### <Receiving Circuitry>

As explained with reference to FIG. 10 of the present disclosure, apparatus 10000 may comprise a receiving circuitry 10002 configured to receive information indicative of a route for a journey.

The receiving circuitry 10002 may be configured as a network connection 1008 which enables the apparatus 10000 to receive information over a network regarding the route for a journey. In fact, the information indicative of the route for the journey can be received in any suitable way (including via both wired and wireless connections). Alternatively, the receiving circuitry 10002 may be configured as a user input device 1006 such that the information can be received directly from a user.

In some examples, the information indicative of the route for a journey may provide information regarding a start point and an end point of the journey. Alternatively, in other examples, the information indicative of the route for the journey may only provide information regarding an end point of the journey (i.e. a destination). Then, information regarding the current location of apparatus 10000 may be used in order to identify the start point for the journey. Further alternatively, the information indicative of the route for the journey may detail the specific path that will be taken during the journey when travelling from the start point to the end point. In other situations, however, the specific path that will be taken during the journey may be identified once the start point and the end point have been provided. This can be determined in any suitable way, such as identifying a path from the start point to the end point which provides the shortest travel time, for example. Therefore, the information regarding the route for the journey may indicate the actual route which will be taken or may indicate certain information which enables the actual route to be determined.

Consider, now, the example of FIG. 11 of the present disclosure. In this example, a person is planning on conducting a journey from an initial start point 11000 to a final end point 11002. The person in this example is planning on travelling by car. During this journey, the person (or indeed another passenger travelling in the car) is planning to watch an image on a display (such as a display incorporated in a mobile telephone device belonging to the person). For example, the person may be planning on watching an action movie in order to entertain themselves while travelling in the car.

As previously noted, watching an image on a display while travelling may cause the person to experience motion sickness, since there may be conflict between the sense of motion experienced by the person (owing to the motion of the car in this example) and the image which is seen by the person (as the image on the display may remain static, or move in a different way, to the car). Watching the action movie for the entirety of the journey may therefore not be suitable and may cause the person to experience excessive symptoms of motion sickness.

Accordingly, in this example, when starting the journey (or when planning the content for the journey prior to the start of the journey), the receiving circuitry 10002 of apparatus 10000 may receive from the person information indicative of the route for the journey 11004. In this example, the route for the journey may describe the path which will be taken by the car as it travels from the start 11000 to the end 11002.

The receiving circuitry 10002 may receive this information from the person by means of a user input or instruction. That is, in the example of FIG. 11 of the present disclosure, the person may issue a voice-command or voice-instruction which informs the receiving circuitry 10002 of the route that will be taken in the up-coming journey (e.g. the person may inform the receiving circuitry 10002 that they will travel from the start 11000 to the end 11002 via a particular road network).

Alternatively, the person may enter information regarding the intended destination (i.e. the end 11002) into another electronic device such as a satellite navigation device and apparatus 10000 may be configured to receive the information regarding the route for the journey from that another electronic device.

In this manner, the receiving circuitry 10002 of apparatus 10000 receives information indicative of the route for the journey.

Of course, it will be appreciated that the present disclosure is not particularly limited to the example illustrated in FIG. 11 of the present disclosure. For example, while the example of FIG. 11 of the present disclosure relates to a journey in a car, the embodiments of the disclosure may also be applied to situations where the person is travelling by other means (including, for example, situations where the person is travelling by a different type of vehicle such as a train). Moreover, while in this example the receiving circuitry 10002 receives the information regarding the route for the journey at the start of the journey (i.e. before the person begins travelling) the receiving circuitry 10002 may also receive this information (or updates for this information (including, for example, a change of route or direction)) after the person has already begun travelling from the start 11000 to the end 11002.

Once the information indicative of the route for the journey has been received, the receiving circuitry 11002 can pass said information to the detecting circuitry 10004. Alternatively, in some examples, the route for the journey may be stored in a storage circuitry from where it can later be retrieved by the detecting circuitry 10004 of apparatus 10000 as required.

### <Detecting Circuitry>

Furthermore, as explained with reference to FIG. 10 of the present disclosure, apparatus 10000 may comprise a detecting circuitry 10004 configured to detect at least a first portion of the journey having a first predetermined characteristic.

Once the route for the journey is available, the detecting circuitry 10004 can use this information in order to identify a portion of the journey which is most likely to cause the person conducting the journey to experience motion sickness. This can be performed by detecting a portion of the journey with a predetermined characteristic which is likely to cause the person to experience motion sickness.

Returning to the example of FIG. 11 of the present disclosure, a person is travelling by car from start 11000 to end 11002 while watching an image on a display device. Detecting circuitry 11004 may know, from the information received by receiving circuitry 11002, that the person will travel from the start 11000 to the end 11002. Indeed, the route the person will take in the vehicle 11004 when travelling from the start 11000 to the end 11002 may be known by the detecting circuitry 10004.

In this example, the detecting circuitry 10004 may detect portions of the journey for which the road is very winding. A long and winding road may be more likely to cause the person to experience motion sickness, since the discrepancy between the sense of motion felt by the person (e.g. from signals from their inner ear) and the movement (or lack thereof) seen by the person when looking at a display device in the car will be greater. On this basis, detecting circuitry 10004 of apparatus 10000 may detect that portion 110012 and 11014 of the journey are portions of the journey 11004 for which the person is most likely to experience motion sickness.

In contrast, when the car is at location 11006 of the journey 11004, the person is very unlikely to experience motion sickness since the car is travelling along a particularly straight portion of the road.

By analyzing the route that will be taken by the car, the detecting circuitry 10004 is thus able to detect portions of the journey most likely to cause motion sickness for a person travelling in the car.

In some examples, where the information indicative of the journey merely indicates an intended route (e.g. that the car should travel from the start 11000 to the end 11002 via a route with the shortest travel time) the detecting circuitry 10004 may detect the portions of the journey having a predetermined characteristic (and thus being most likely to cause motion sickness) by comparing the journey with an additional source of information such as mapping information or the like. Portions of the journey with a predetermined characteristic (e.g. very winding portions of the journey) can then be detected from the mapping information.

As such, it will be appreciated that the detecting circuitry is configured to detect the first portion of the journey based on the information indicative of the route for the journey and/or one or more sources of additional information (such as mapping information or the like).

Of course, it will be appreciated that while in the example described with reference to FIG. 11 the predetermined characteristic relates to how windy the road is, the present disclosure is not particularly limited in this regard. That is, any characteristic of the journey which can be used to detect portions of the journey which are most likely to cause the person to experience motion sickness can be used in accordance with embodiments of the disclosure. This may vary depending on the context of the situation to which embodiments of the disclosure are applied.

For example, when the person is to travel by plane, the detecting circuitry 10004 of apparatus 10000 may detect portions of the journey which are most likely to have significant levels of turbulence as portions of the journey which are most likely to cause the person to experience motion sickness (that is, a predisposition of that portion of the journey to turbulence can be used as the first predetermined characteristic in accordance with embodiments of the disclosure). Information regarding the likelihood of turbulence during the journey may be detected based on the geography of the land over which the plane will fly (e.g. whether the plane will fly over warm waters or close to mountains).

Alternatively, such information regarding the likelihood of turbulence may be determined based on a comparison of the route for the journey with a weather forecast (e.g. a portion of the journey coincident with an area of strong winds or atmospheric disturbance).

Alternatively, when the person is to travel by plane, a predetermined characteristic of the journey may also include portions of the journey related to take-off or landing procedures. These portions of the journey may also lead to higher levels of motion sickness in a user owing to the increased amount of movement.

Further alternatively, the predetermined characteristic of the journey may include portions of the journey with rapid changes in elevation (e.g. such as when driving along a bumpy road), the type of road being traversed at a given stage of the journey (e.g. motorways are often straighter and less likely to cause motion sickness than a country lane), a portion of the journey for which accelerations will be higher (including, for example, portions of the road with frequent speed changes), a portion of a journey with a sequence or pattern of motion (e.g. a route with a particular sequence of corners, such as those illustrated in portion 11014 of journey 11004) or the like. In fact, the predetermined characteristic of the portion of the journey may also relate to historic information regarding motion sickness experienced by the person or other people who have travelled the same route. That is, if the historic data indicates that a particular portion of the journey leads to a high incidence of motion sickness, then that portion of the journey may be detected by the detecting circuitry 10004.

The present disclosure is not particularly limited to these examples of the predetermined characteristic. Many other types of predetermined characteristic can be used depending on the type of journey which will be taken.

As such, it will be appreciated that the detecting circuitry 10004 of the present disclosure may use any predetermined characteristic of the route when detecting portions of that journey as appropriate depending on the situation to which the embodiments of the disclosure are applied, insofar as the predetermined characteristic can be used to detect portions of the journey most likely to cause the person travelling along the route to experience motion sickness.

### <Identifying Circuitry>

As explained with reference to FIG. 10 of the present disclosure, apparatus 10000 may comprises an identifying circuitry 10006 configured to identify a time period corresponding to the first portion of the journey.

Once a particular portion (or particular portions) of the journey have been detected as those portions of the journey which, when traversed, are most likely to cause a person to experience motion sickness, this portion (or portions) of the journey can be passed to the identifying circuitry 10006 of apparatus 10000 such that apparatus 10000 can identify the actual time during the journey the portion (or portions) will be reached. By identifying the actual time during the journey the portion (or portions) will be reached, it is possible for apparatus 10000 to carefully select content for display to the user at that time which is sensitive to the fact that the user may be more likely to be experiencing motion sickness. In this manner, motion sickness of the user can be reduced.

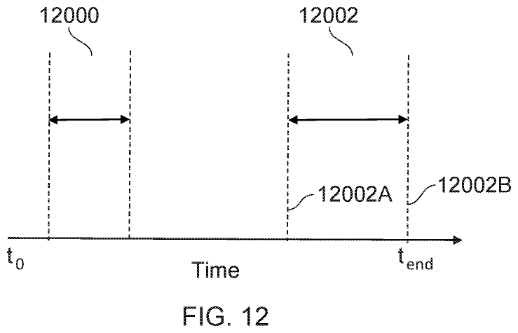

Consider, now, FIG. 12 of the present disclosure. This example illustrates a timeline in accordance with embodiments of the disclosure. That is, the timeline in FIG. 12 of the present disclosure illustrates the total amount of time an example journey would take to complete. The timeline begins at time to (corresponding to the start of the journey) and progresses until t_{end} (which corresponds to the end of the journey). The unit of time used for the timeline is not particularly limited and will vary depending on the type of journey. However, for a typical car journey (such as that illustrated in FIG. 11 of the present disclosure) the timeline may be formed in a unit of time such as hours and/or minutes. A timeline in seconds can also be used, for example.

The timeline for the journey (such as that illustrated in FIG. 12 of the present disclosure) may be generated once the route for the journey has been received by the receiving circuitry. The timeline may, for example, be estimated or constructed based on information from one or more additional sources of information (such as mapping information, or the like). Indeed, in some examples, the timeline for the journey may be estimated or constructed in accordance with contextual information including the time of day, the type of roads included in the journey and/or the level of traffic or congestion currently present on the route. An example journey (such as that illustrated in FIG. 11 of the present disclosure) may have a first timeline if conducted by a person during the afternoon, however that same that same journey may have a second, different, timeline if the journey is conducted by the person during a very busy time with lots of traffic (such as early in the morning, when more people may be travelling to work).

Moreover, the timeline for the journey may be updated as the journey progresses. That is, the estimated journey duration may be either shortened or lengthened by apparatus 10000 depending on how the person's journey is progressing. If the person takes an unscheduled stop on route or encounters some unexpected traffic on the road the timeline of the journey (and thus the estimated journey duration) may be increased.

Once constructed, the timeline of the journey can be correlated with the route (e.g. using mapping information or the like) in order to identify at what time during the journey the person will reach the portions of the journey which have been detected by the detecting circuitry 10004.

In the example of FIG. 12 of the present disclosure the period of time identified as 12000 may correspond to the portion of the journey 11012 identified in FIG. 11 of the present disclosure as a first portion of the journey likely to cause motion sickness (i.e. having the first predetermined characteristic). Furthermore, the period of time identified as 12002 may correspond to the portion of the journey 11014 identified in FIG. 11 of the present disclosure as the second portion of the journey likely to cause motion sickness (i.e. the second portion of the journey having the first predetermined characteristic). The time 12002A may thus correspond to the time at which it is estimate the car will reach location 11008, while the time 12002B may correspond to the time at which it is estimated that the car will reach location 11010. The time period 12002 between time 12002A and 12002B is therefore the amount of time that it is estimated it will take for the car to travel between 11012 and 11014 in the example of FIG. 11 of the present disclosure.

In this manner, the identifying circuitry 10006 of apparatus 1000 can identify a time period corresponding to a portion of the journey having the first predetermined characteristic. Apparatus 10000 will then know when the person who is travelling will reach the portion of the journey having the first predetermined characteristic and also how long that portion of the journey will last once it has been reached. As an example, apparatus 10000 may know that portion 11014 will be reached at 10.15am, and the person will take 20 minutes to travel from 11008 to 11010. Of course, these times are only an example and are not limiting to the present disclosure.

It will be appreciated that identifying a time period corresponding to a portion of the journey having the first predetermined characteristic in this manner is particularly advantageous. That is, by identifying the time during the journey at which the portions of the journey most likely to cause motion sickness will occur and by identifying how long each of these portions will last, apparatus 10000 is able to construct a plan in advance of the journey for the most appropriate content to be displayed on a display device within the vehicle at each stage of the journey such that motion sickness experienced by the user during the journey can be alleviated. This improves efficiency of processing, since the content for display can be obtained in advance.

Of course, as previously explained, the timeline may be updated or revised during the journey (based on current location information of the car, for example). However, such adjustments may typically require only a small modification of the timeline and the corresponding content which is to be displayed (e.g. a small delay before reaching portion 11014 of the journey (such as an unexpected stop) may delay the start of the time period 12002 but may not actually increase the duration of the time period 12002 (i.e. the time to traverse 11014 once that portion has been reached). Therefore, even if the timeline is updated or revised during the journey, changes to content selected for display may be quite small and efficiently performed.

Identifying circuitry 10006 may identify the time period corresponding to the portion or portions of the journey having the first predetermined characteristic in any suitable way depending on the situation. That is, in some examples, these time periods may be identified without constructing a timeline for the entire journey. Considering the example of FIG. 11 of the present disclosure, the identifying circuitry 10006 may identify that the portion of the journey identified as 11012 may last for a period of 15 minutes (or indeed any other period of time) based merely on an analysis of that portion of the journey (e.g. the length of that portion of the road and the predicted speed of the car when travelling along that portion of the road). Then, when is detected that the car reaches that portion of the road (based on location information of the car) appropriate predetermined content having a duration of 15 minutes and which has been selected by the selecting circuitry 10008 of apparatus 10000 in advance may be displayed to the person—regardless of when during the journey (e.g. how long since the start of the journey) that portion of the journey is reached. Therefore, motion sickness can be alleviated even when only the time periods corresponding to the portion or portions of the journey having the predetermined characteristic are identified (as opposed to the construction of an entire timeline for the journey).

### <Selecting Circuitry>

As explained with reference to FIG. 10 of the present disclosure, apparatus 10000 may comprise a selecting circuitry 10008 configured to select one or more items of content having a second predetermined characteristic for display over the time period.

As noted in the Background, a sense of motion sickness is most likely to be experienced by a person when there is discord between the different senses of the person. In particular, discord between the sense of motion caused by a car travelling along a specific route (as sensed by a person's inner ear) and the motion (or lack thereof) seen by the person's eyes can cause motion sickness in the person. Differences between these signals may be exacerbated when the user is watching certain types of content on a display device.

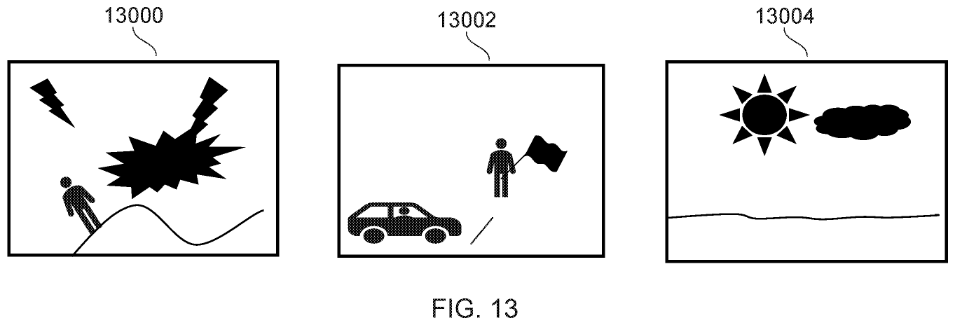

Consider, now, the example of FIG. 13 of the present disclosure. FIG. 13 illustrates an example of content with different characteristics in accordance with embodiments of the disclosure.

In this example, three pieces of content are shown; content 13000, content 13002 and content 13004. Each of these pieces of content represents a particular piece of video content. That is, while a single image frame is shown for each piece of content, it will be appreciated that each piece of content may actually comprises a number of different image frames which can be displayed to a user in a video sequence.

In this example, the first piece of content 13000 represents an action film (or movie). In this action film, there are a may be a large number of different computer generated effects. Moreover, there may be number of different objects moving relative to each other between image frames of the film or video sequence. Therefore, a person watching the first piece of content 13000 will see with their eyes lots of different visual indicators of movement. If a person is watching this first piece of content during a journey such as that illustrated in FIG. 11 of the present disclosure, then the person may be more likely to experience a sense of motion sickness (as the risk of conflict between the motion seen by the person in the film and the sense of motion experienced by the person as the car travels along the road increases).

Furthermore, in this example, the second piece of content 13002 represents a video such as a television programme showing a motor sport. In this television programme, a number of racing cars may be shown driving very quickly around a race track in an attempt to reach the finish line first. Therefore, a person watching the second piece of content 13002 will see with their eyes lots of different visual indicators of movement. If a person is watching this second piece of content during a journey such as the journey illustrated in FIG. 11 of the present disclosure, the person may be more likely to experience a sense of motion sickness (as the risk of conflict between the motion seen by the person in the television programme and the sense of motion experienced by the person as the car travels along the road increases).

Finally, in this example, the third piece of content 13004 represents a video such as a relaxing film showing a sequence of images of a beach. In this video content, there is very little sense of motion between the different image frames of the video. Moreover, watching the video may provide the user with a sense of calm relaxation. Therefore, a person watching this third piece of content 13004 will see with their eyes very few visual indicators of motion and will experience a sense of calm relaxation. If a person is watching this third piece of content during a journey such as the journey illustrated in FIG. 11 of the present disclosure, then the person may be less likely to experience a sense of motion sickness (as the risk of conflict between the motion seen by the user in the video content and the sense of motion experienced by the person as the car travels along the road decreases).

The third piece of content 13004 is therefore a piece of content having a second predetermined characteristic, the second predetermined characteristic being any characteristic which indicates that a user watching the content is less likely to experience a sense of motion sickness. That is, in this example, the characteristic of being content with very few visual indicators of motion is the second predetermined characteristic, as content with very few visual indicators of motion will be less likely to cause the user to experience a sense of motion sickness. However, this is only one type of second predetermined characteristic which can be used in accordance with embodiments of the disclosure.

It will be appreciated that, generally, the second predetermined characteristic is a characteristic of the content indicative of the likelihood of an item of content to cause motion sickness. In some examples, items of content having the second characteristic are items of content which are less likely to cause and/or will reduce or alleviated a sense of motion sickness in a user. Indeed, while the level of movement in content has been described, with reference to FIG. 13 of the present disclosure, as an example of the second characteristic of the content, it will be appreciated that the present disclosure is not particularly limited in this regard. For example, the second predetermined characteristic may also include an indicator of the type of content (e.g. whether the user will find the content stressful or relaxing), the number of scene changes in the content, a measure of the amount of text in the content, a historic indicator of whether the content is likely to cause a sense of motion sickness in the user, or the like.

Of course, it will be appreciated that the present disclosure is also not particularly limited to the very specific example of different items of content illustrated in FIG. 13. Rather, there are many different types of content which can be used in accordance with embodiments of the disclosure, including different types of images, videos, text, video games, user interfaces, internet web pages or the like. Indeed, any type of content which can be displayed on a display device can be used in accordance with embodiments of the disclosure.

The different types of content may be stored locally to apparatus 10000. Alternatively, the different types of content may be stored externally to apparatus 10000 (such as on a server or database) and accessed by apparatus 10000 as required.

In some examples, apparatus 10000 may be configured to perform processing steps on the content in order to enhance the second characteristic of the content (and thus make the content even more suitable for reducing or alleviating a sense of motion sickness in the user). The type of processing which is performed by apparatus 10000 in order to enhance the second characteristic of the content is not particularly limited and depends on the type of content and the second characteristic of that content itself. However, in the example of FIG. 13 of the present disclosure (where the second characteristic relates to the amount of movement within the video content) apparatus 10000 may enhance the second characteristic of a piece of content, such as content 13004, by processing the image frames of the video content in order to remove any objects within the content which are moving rapidly in the scene, processing the content in order to reduce the he speed of transition between video frames, performing processing to skip over a portion of the content which contains objects which are moving excessively quickly between frames in the video content, or the like. Of course, the processing performed to enhance the second characteristic of the content is not particularly limited to these examples and other types of processing can be performed as required.

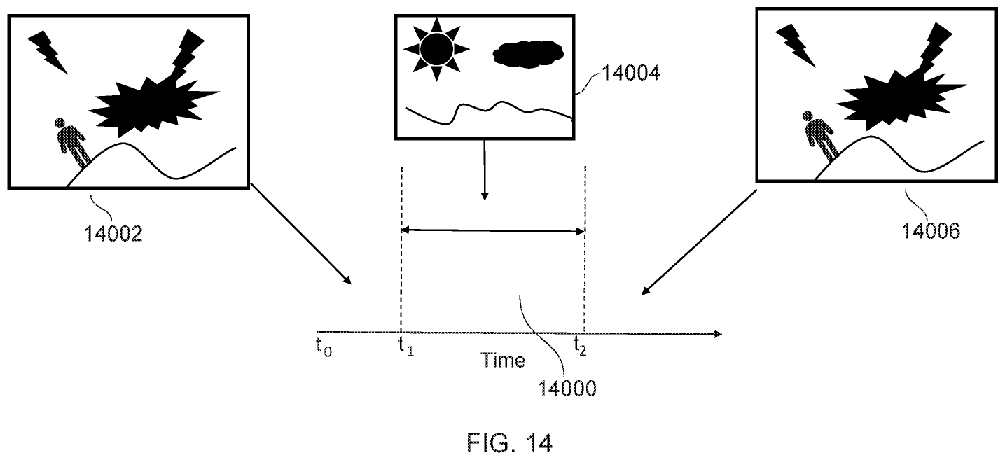

Consider, now, FIG. 14 of the present disclosure. In this example, a timeline in accordance with embodiments of the disclosure is illustrated. The timeline of FIG. 14 is similar to the timeline described with reference to FIG. 12 of the present disclosure. However, the timeline of FIG. 14 of the present disclosure corresponds to a journey for which only a single portion has been detected by the detecting circuitry as having a first predetermined characteristic.

The timeline of FIG. 14 of the present disclosure starts at to (being, in this example, the time at which the journey is started). The part of the journey from t₀ to t₁ has not been identified as a part of the journey which is likely to cause motion sickness in the user. However, the time period 14000 (from t₁ to t₂) has been identified by apparatus 10000 as a part of the journey which is most likely to cause motion sickness in a person watching a display during the journey. This may, for example, be because the part of the road being traversed during this portion of the journey is particularly windy (with lots of corners and turns in the road).

The part of the journey after time t₂ has not been identified as being a part of the journey likely to cause motion sickness.

Based on this information, the selecting circuitry 10008 can therefore select one or more items of content having a second predetermined characteristic for display over the time period 14004. The second predetermined characteristic used to select the one or more items of content for display is a characteristic of the content which indicates that the content is less likely to cause and/or may be used in order to alleviate a sense of motion sickness in a person. Taking the example items of content described with reference to FIG. 13 of the present disclosure as an example, the second predetermined characteristic of the content may be a characteristic indicative of the amount of movement within the content—with content with a lower amount of movement being less likely to cause motion sickness in the person. Accordingly, of the example pieces of content 13000, 13002 and 13004, the content 13000 and 13002 may be identified as content with high amounts of movement in the content while content 13004 may be identified as content with low amounts of movement. Therefore, content 13004 is a piece of content with the second predetermined characteristic (e.g. lower amounts of movement and less likelihood of causing motion sickness). Selecting circuitry 10008 may therefore select content 13004 as content which is suitable for display during the portion of the journey 14000 (between time t₁ and t₂)—being the portion of the journey which is most likely to cause a person to experience motion sickness. Indeed, by selecting this content for display during this period, apparatus 10000 can plan appropriate content for the journey such that a person's sense of motion sickness may be reduced. Specifically, by displaying content 14004 during the time period 14000 a person's sense of motion sickness during the portion of the journey most likely to cause motion sickness may be reduced or alleviated.

In the example of FIG. 14 of the present disclosure, for the time period between t0 and t1 and the time period after t2 (until the end of the journey) apparatus 10000 may choose to display any type of content. This is because these parts of the journey are parts of the journey which are much less likely to cause motion sickness in a person. As such, content such as content 13000 of FIG. 13 of the present disclosure may be selected for display during these periods. In some examples, the content selected for display in periods other than 14000 (i.e. those periods of time which are not linked to particularly motion sickness inducing parts of the journey) may be selected based on user preferences or user commands. Then, only when the part of the journey corresponding to the time period 14000 (i.e. from t₁ to t₂) would the content 14004 be displayed in order to reduce the feeling of motion sickness in the user.

In some examples of the disclosure, once the selecting circuitry 10008 of apparatus 10000 has selected the content which is suitable for display, apparatus 10000 may further cause a display device to display this content at the predetermined time during the journey. That is, apparatus 10000 may issue a control command or the like which instructs the display device to display the content which has been selected by the selecting circuitry 10008 at the appropriate time during the journey. As such, in some embodiments of the disclosure, apparatus 10000 may be configured to display the one or more items of content having the second predetermined characteristic during the time period corresponding to the first portion of the journey.

As an example, a person travelling in the car travelling from the start 11000 to the end 11002 may decide that they wish to watch an action movie during the journey. Accordingly, the display in the car may show this action movie as the car is travelling from the start 11000 to the end 11002. However, when the car reaches the portion 11012 or the portion 11014, the apparatus 10000 may control the display to warn the user that they are entering a portion of the journey most likely to cause motion sickness. Moreover, apparatus 10000 may then control the display in order to display certain content on the display (such as content 13004) to alleviate or reduce the sense of motion sickness experienced by the user. Once the car exits portion 11012 or 11014 and is thus travelling on a portion of the road less likely to cause motion sickness, apparatus 10000 may control the display such the action movie is again shown on the display. In this way, a person's sense of motion sickness when travelling can be reduced.

As noted above, the selecting circuitry 10008 selects one or more items of content from an internal or external storage device for display during a time period which has been identified as a time period corresponding to a portion of the journey which is particularly likely to cause or induce a feeling of motion sickness in the user. The content which is selected by the selecting circuitry 10008 is content with a second predetermined characteristic (being a characteristic which is likely to alleviate or reduce the sense of motion sickness in the user). The selected content is content which is suitable for display during the time period. However, there may be a situation whereby the content which is available for selection by the selecting circuitry is has a different temporal length (or duration) than the time period for which the content should be displayed. That is, there may be a mismatch between the time period for which content should be selected and the duration of the content having the second predetermined characteristic and which is available for selection. This may be a particular problem when the content which is available for selection has a shorter duration that the time period. In this situation, content which can reduce or alleviate the sense of motion sickness in the user may not be available for display for the entire time period of the journey which has been identified.

Accordingly, in some examples, apparatus 10000 may be configured to determine a difference between the length of the time period and the duration of the one or more items of content; and modify the route for the journey to reduce the difference when the difference is above a predetermined threshold.

Consider, again, the example of FIG. 11 of the present disclosure. In this example, a portion of the journey 11014 has been identified as a portion of the journey which is likely to induce a sense of motion sickness in the person travelling in the car. Accordingly, a time period corresponding to this portion of the journey is identified; in this example, portion 11014 of the journey may last for duration of 20 minutes. Therefore, the selecting circuitry 10008 may select one or more items of content suitable for display during this time period. The content display during the portion 11014 may last for 20 minutes (so that it can be displayed for the entire time that the car is travelling through portion 11014 of the journey) and may have a second predetermined characteristic (being a characteristic that is suitable for reducing or alleviating a sense of motion sickness in the user). Of the content available for selection, the selecting circuitry 10008 may identify that a piece of content 13004 has the second predetermined characteristic. However, this content 13004 may have a duration of only 19 minutes. Accordingly, the difference between the time period and the duration of the content 13004 is 1 minute. In this example, the predetermined threshold which triggers modification of the route for the journey may be a difference of 30 seconds or more. Therefore, since the difference between the time period and the duration of the content (i.e. 1 minute) is above this threshold (i.e. 30 seconds) the selecting circuitry 10008 of apparatus 10000 may modify the route for the journey in order to reduce the difference between the time period and the duration of the content. In this example, selecting circuitry 10008 may identify that a slightly different route through portion 11014 of the journey is possible, which has a slightly shorter time period than the original route which had been selected. This may be identified be comparison of the route with mapping information or the like. This slightly shorter route may have a duration of 19 minutes and 20 seconds. Accordingly, by taking this alternative route, the difference between the time period and the duration of the content can be reduced such that content suitable for reducing or alleviating the sense of motion sickness in the person can be displayed for an increased percentage of the time when the car is travelling through portion 11014. This ensures that a sense of motion sickness in a person can be more effectively and efficiently reduced during the journey.

Alternatively or in addition, when the difference between the length of the time period and the duration of the one or more items of content is above the predetermined threshold, apparatus 1000 may be configured to modify the one or more items of content in order to reduce the difference when the difference is above a predetermined threshold.

In the example described with reference to FIG. 11 of the present disclosure (where the difference between the time period and the duration of the content is 1 minute, but the predetermined threshold is 30 seconds), apparatus 10000 may modify the content 13004 in order to reduce the difference between the duration of this content and the time period 11014. For example, apparatus 10000 may modify a playback speed of the content such that the content has a duration which is the same (or much closer) to the time period. Displaying content 13004 with a playback speed of 0.95 (compared to its intended or original speed) will mean that content 13004 will last for a duration of 20 minutes. Therefore, the content 13004 which can reduce or alleviate the sense of motion sickness in a user can be displayed for the entirety of the time when the car is travelling through portion 11014. Moreover, such a modification of the playback speed of the content may not be perceptible to the person when the change in the playback speed of the content is within certain limits. By reducing the difference in the between the duration of the content and the time period 11014, a sense of motion sickness in a person can be more effectively and efficiently be reduced during the journey.

However, it will be appreciated that modification of the one or more items of content which have been selected by the selecting circuitry 10008 in order to reduce the difference between the time period and the duration of the content is not limited to modification of the playback speed of the content. In some examples, when the difference between the duration of the content and the time period is above a predetermined threshold, apparatus 10000 may be configured to generate an additional item of content for display during the time period once the one or more selected items of content have been displayed. That is, if no further suitable items of content are available for selection, apparatus 10000 may generate an additional item of content having the second predetermined characteristic which can be used for display in the time remaining while the car is travelling through the portion 11014 once the selected items of content have been displayed. In the example described with reference to FIG. 11 of the present disclosure, if the difference between the time period and the duration of the content 13004 is 1 minute, then apparatus 10000 may generate an additional item of content suitable for display while the car is travelling through the portion 11014 of the journey which has a duration of 1 minute. This can then be displayed once the selected items of content have been displayed in order to ensure that content suitable for reducing or alleviating the sense of motion sickness of a person can be displayed for the entire time during which the car is travelling through portion 11014.

The additional item of content can be generated by apparatus 10000 based on the items of content which are already available within the internal or external storage. For example, apparatus 1000 may be configured to generate a short summary, highlight or trailer relating to an existing item of content as the additional item of content. Alternatively, apparatus 10000 may generate an additional item of content by selecting an image frame from a piece of video content and generating a new video content based on that image such that the image will be displayed to the user for the appropriate amount of time. Alternatively, the new items of content may include computer generated items of content or items of content produced by machine learning techniques or the like. In some examples, the additional item of content which is generated by apparatus 10000 may include an advertisement having the second predetermined characteristic (being that it is suitable for reducing or alleviating the sense of motion sickness in the user). This advertisement can be shown for the time remaining once the selected content has been displayed to the user when travelling through portion 11014. Once the advertisement ends, the user can return to watching any type of content (since they will no longer be within a portion of the journey which is likely to cause motion sickness).

It will be appreciated that the present disclosure is not particularly limited to these examples which have been described with reference to FIG. 11 of the present disclosure. Time differences much longer and much shorter than those described in this example may be used in accordance with embodiments of the disclosure. Moreover, different types of content, different modifications of the route for the journey and/or different modifications of the content which have been selected can be performed by apparatus 10000 as required depending on the situation to which the embodiments of the disclosure are applied.

Now, it will be appreciated that the portions of the journey which have been detected by the detecting circuitry 10002 are those portions of the journey which are most likely to cause motion sickness in the user. Generally, these are any portions of the journey which have the first predetermined characteristic. If a portion of the journey has this predetermined characteristic (e.g. the portion of the journey relates to a very winding section of the road) then apparatus 10000 may select content which is suitable for display during this portion of the journey. The content which is selected will be content which, when watched by a person on a display device, will reduce or alleviate the sense of motion sickness of the user.

However, in some examples, certain portions of a journey may have a very strong likelihood of causing motion sickness in a user (e.g. a likelihood of causing motion sickness above the level indicated by the first predetermined characteristic). Consider, again, FIG. 11 of the present disclosure. In this example, portion 11014 of the journey comprises lots of sharp turns in short succession. Therefore, portion 11014 of the journey has a very strong likelihood of causing a sense of motion sickness in the user. If a portion of the journey has a third predetermined characteristic which is indicative of a very high likelihood of motion sickness (such as the rapid succession of turns illustrated in FIG. 11) then the apparatus 10000 and/or selecting circuitry 10008 may make a different selection of content for that time period. A very high likelihood of motion sickness may be a likelihood of motion sickness above a predetermined threshold. The predetermined threshold may be set based on the user, the journey or the type of vehicle being used for the journey. A portion of the content having the third predetermined characteristic (indicative of a very high likelihood of motion sickness) may also relate to a sub-portion of a portion of a journey having a second predetermined characteristic (indicative of a likelihood of motion sickness).

In some examples, apparatus 10000 may be configured to pause the display of content during a time period relating to a portion of the journey with a very high likelihood of causing motion sickness in the person. By pausing the display of content, the person will then stop watching content on the display which may further reduce or alleviate the sense of motion sickness during the portions of the journey with a very high likelihood of causing motion sickness. Moreover, additional content such as a warning message (e.g., “warning, high likelihood of motion sickness occurring”) and/or instructions to the user of how to alleviate motion sickness (e.g., “stop looking at the display during the next portion of the journey”) may be generated and displayed on the display while the original content has been paused in order to further encourage the person to stop watching the display while travelling through the portions of the journey most likely to cause motion sickness.

Once the person has travelled through these worst sections of the journey (i.e. the portions of the journey having the third predetermined characteristic, indicating that the likelihood of the user experiencing motion sickness during that portion of the journey is above a predetermined threshold) then apparatus 10000 may resume the playback of content. That is, any content which was being displayed before the playback of content was paused may continue to be displayed once that portion of the journey is over. Therefore, even in the portions of the journey which are most likely to cause motion sickness in the user (being those portions with the third predetermined characteristic) the selection of content for display during the journey can be made such that the sense of motion sickness in the user can be alleviated or reduced. As such, for sections of the journey, such as 11006 of FIG. 11, the user may be free to watch any content on the display. Then, for sections of the journey having a first predetermined characteristic, specific content for reducing or alleviating the feeling of motion sickness in the user may be displayed. However, for sections of the journey with the third predetermined characteristic (being the sections of the journey where the likelihood of experiencing motion sickness is above a predetermined threshold) the display of content on the display device may be paused such that the user stops watching the display during that time. The feeling of motion sickness of the user during the journey can therefore be effectively and efficiently reduced.

### <Advantageous Technical Effects>

The apparatus 10000 provided in accordance with embodiments of the disclosure (as described with reference to FIGS. 10 to 14 of the present disclosure) is configured to select content for display during a journey to alleviate motion sickness. That is, when a portion of a journey with a first predetermined characteristic (such as a portion of the journey which is likely to cause motion sickness) has been identified, apparatus 10000 is able to select content which is most suitable for this portion of the journey. Accordingly, content which is likely to reduce the feeling of motion sickness can be displayed during the portions of the journey which are most likely to make the person feel unwell. Motion sickness during the journey can therefore be alleviated or reduced.

Of course, it will be appreciated that the advantageous technical effects provided by embodiments of the disclosure are not particularly limited in this regard. Other advantageous technical effects will become apparent to the skilled person when reading the disclosure.

### <Method>

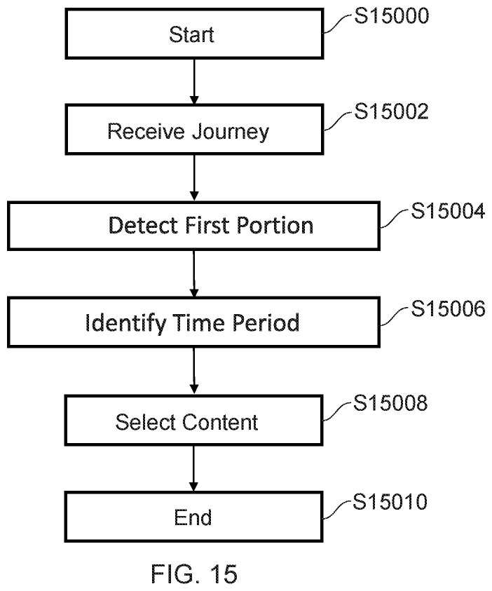

FIG. 15 illustrates an example method in accordance with embodiments of the disclosure. The method is a method of selecting content for display during a journey to alleviate motion sickness. This method may be performed by an apparatus such as apparatus 1000 of the present disclosure, for example. Furthermore, a computer program product comprising instructions which, when implemented by a computer, cause the computer to perform the method illustrated in FIG. 15 of the present disclosure may be provided.

The method of FIG. 15 starts at S15000 and proceeds to step S15002. The start of the method may, in some examples, be a time prior to the start of the journey. However, the present disclosure is not particularly limited in this regard and the start of the method may correspond to any other time depending on the situation.

In step S15002, the method comprises receiving information indicative of a route for a journey. The route for the journey may, in some example, be the route which will be taken by a vehicle such as a car when travelling from one point to another. Once the information indicative of the route for the journey has been received, the method proceeds to step S15004.

In step S15004, the method comprises detecting at least a first portion of the journey having a first predetermined characteristic. The first portion of the journey may, in some examples, be a portion—such as portion 11014 of the example of FIG. 11 of the present disclosure—which is particularly likely to induce a feeling of motion sickness. The method then proceeds to step S15006.

In step S15006, the method comprises identifying a time period corresponding to the first portion of the journey. The time period corresponding to the first portion of the journey may indicate a period of time during a predicted timeline of the journey that will correspond to the first portion of the journey. Moreover, the time period may indicate how long the first portion of the journey will last for once it occurs (i.e. the duration of the first portion of the journey). The method then proceeds to step S15008.

In step S15008, the method comprises selecting one or more items of content having a second predetermined characteristic for display over the time period. The content may, for example, have a particular characteristic which is likely to reduce the sense of motion sickness experienced by a user. Therefore, content suitable for the first portion of the journey (being, for example, a portion of the journey most likely to induce a sense of motion sickness in a user) can be selected in advance.

The method then proceeds to and ends with step S15010. In some examples, however, the method illustrated in FIG. 15 of the present disclosure—or individual steps within that method—may be repeatedly performed. That is, for example, even once content has been selected for the first portion of the journey, the content may be updated or modified in accordance with changes or delays in the journey. Moreover, other method steps may be included in this method as appropriate. Indeed, these method steps do not have to be performed in the order illustrated in FIG. 15 and a number of the method steps of FIG. 15 may be performed in parallel with respect to one or more of the other method steps illustrated in FIG. 15.

While a number of embodiments of the disclosure have been described with reference to the example of motion sickness arising in the context of the motion of a car, it will be appreciated that the present disclosure is not particularly limited in this regard. Rather, the embodiments of the present disclosure may be applied in order to alleviate the sense of motion sickness arising from watching a display device in any situation. This may include watching a display device when travelling in other types of vehicles including, for example, travelling on any other type of road vehicle (e.g. a bus) or travelling on a vehicle such as a plane or a boat. Furthermore, embodiments of the present disclosure may also be applied to situations where a user is watching a display device when not travelling on a vehicle (e.g. when walking). The advantageous technical effects of reducing the sense of motion sickness can be achieved in all of these situations.

Moreover, it will be appreciated that the first embodiment of the disclosure and the second embodiment of the disclosure are not mutually exclusive. That is, elements of the first embodiment (regarding the dynamic compensation of motion) and elements of the second embodiment of the disclosure (regarding planning and selecting content for display during a journey) may be combined in order to further reduce the sense of motion sickness experienced by a user. For example, apparatus 10000 may select content for display during the journey in order to reduce or alleviate the sense of motion sickness of the user. However, when travelling, this content which has been selected can be displayed by apparatus 3000 such that the image location on a display device is dynamically adjusted in accordance with the movement experienced during the journey. This further ensures that the sense of motion sickness experienced by a user can be most efficiently and effectively reduced.

In addition, embodiments of the present disclosure may be arranged in the manner defined by the following numbered clauses:
1. A method of alleviating motion sickness in a user viewing an image on a display, the method comprising:
displaying an image on a display device, the display device being a first distance from a user viewing the image on the display device;
generating a virtual screen corresponding to the display device by projecting a line from the user and the display device to a second location, the distance of the second location from the user being greater than the first distance;
detecting a movement of the user and the display device to a new location relative to the virtual screen;
determining a new image location by projecting a line from the virtual screen location to the new location of the user;
and displaying an image on the display device at the new image location for at least a portion of the image for which the new image location intersects with the display device.
2. The method according to clause 1, wherein the user and the display device are on-board a vehicle and wherein the method comprises detecting a movement of the user and the display device by detecting a movement of the vehicle.
3. The method according to clause 2, wherein the distance from the user to the display device remains the first distance when the user and the display device move to a new location relative to the virtual screen.
4. The method according to clause 2, wherein the method comprises detecting the movement of the vehicle using one or more of gyroscopic sensors, accelerometer sensors, GPS signals, mapping information and/or control signals for controlling the motion of the vehicle.
5. The method according to any preceding clause, wherein the movement of the user and the display device includes a rotational and/or translational movement.
6. The method according to any preceding clause, wherein the image comprises a photograph, text content and/or a frame from a video.
7. The method according to any preceding clause, wherein the method comprises generating audio associated with the image when the image is displayed on the display device; and move a source location of the audio in accordance with the new image location when displaying the image on the display device at the new image location.
8. The method according to any preceding clause, wherein the method comprises adapting the display of the image on the display device when the movement of the user and the display device relative to the virtual screen is above a threshold value.
9. The method according to clause 8, wherein adapting the display of the image on the display device includes displaying a predetermined image to replace the image on the display device.
10. The method according to clause 9, wherein the predetermined image includes a blank image and/or an image with a predetermined property.
11. The method according to clause 8, wherein the method comprises generating audio associated with the image when the display of the image on the display device has been adapted.
12. The method according to clause 8, wherein when the movement of the user and the display device relative to the virtual screen remains above the threshold value for a predetermined amount of time, the method comprises moving the virtual screen to a new virtual screen location corresponding to the location defined by projecting a line from the new location of the user and the display device to a third location, the distance of the third location being the same as the second location.
13. The method according to clause 12, wherein the method comprises incrementally moving the virtual screen to the new virtual screen location over a second predetermined period of time.
14. The method according to clause 8, wherein the threshold value corresponds to a movement for which no portion of the new image location intersects with the display device.
15. The method according to any preceding clause, wherein when no portion of the new image location intersects the display device, the method comprises adjusting the new image location such that at least a predetermined portion of the image intersects the display device.
16. The method according to clause 15, further comprising adapting the image for display to indicate that the new image location has been adjusted.
17. An apparatus for alleviating motion sickness in a user viewing an image on a display, the apparatus comprising circuitry configured to:
display an image on a display device, the display device being a first distance from a user viewing the image on the display device;
generate a virtual screen corresponding to the display device by projecting a line from the user and the display device to a second location, the distance of the second location from the user being greater than the first distance;
detect a movement of the user and the display device to a new location relative to the virtual screen;
determine a new image location by projecting a line from the virtual screen location to the new location of the user;
and display an image on the display device at the new image location for at least a portion of the image for which the new image location intersects with the display device.
18. Computer program product comprising instructions which, when the instructions are implemented by a computer, cause the computer to perform a method of alleviating motion sickness in a user viewing an image on a display, the method comprising:
displaying an image on a display device, the display device being a first distance from a user viewing the image on the display device;
generating a virtual screen corresponding to the display device by projecting a line from the user and the display device to a second location, the distance of the second location from the user being greater than the first distance;
detecting a movement of the user and the display device to a new location relative to the virtual screen;
determining a new image location by projecting a line from the virtual screen location to the new location of the user;
and displaying an image on the display device at the new image location for at least a portion of the image for which the new image location intersects with the display device.

While a number of embodiments of the disclosure have been described with reference to the example of motion sickness arising in the context of the motion of a car, it will be appreciated that the present disclosure is not particularly limited in this regard. Rather, the embodiments of the present disclosure may be applied in order to alleviate the sense of motion sickness arising from watching a display device in any situation. This may include watching a display device when travelling in other types of vehicles including, for example, travelling on any other type of road vehicle (e.g. a bus) or travelling on a vehicle such as a plane or a boat. Furthermore, embodiments of the present disclosure may also be applied to situations where a user is watching a display device when not travelling on a vehicle (e.g. when walking). The advantageous technical effects of reducing the sense of motion sickness can be achieved in all of these situations.

Moreover, it will be appreciated that the first embodiment of the disclosure and the second embodiment of the disclosure are not mutually exclusive. That is, elements of the first embodiment (regarding the dynamic compensation of motion) and elements of the second embodiment of the disclosure (regarding planning and selecting content for display during a journey) may be combined in order to further reduce the sense of motion sickness experienced by a user.

Obviously, numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1) A method of alleviating motion sickness in a user viewing an image on a display, the method comprising:
displaying an image on a display device, the display device being a first distance from a user viewing the image on the display device;
generating a virtual screen corresponding to the display device by projecting a line from the user and the display device to a second location, the distance of the second location from the user being greater than the first distance;
detecting a movement of the user and the display device to a new location relative to the virtual screen;
determining a new image location by projecting a line from the virtual screen location to the new location of the user;
and displaying an image on the display device at the new image location for at least a portion of the image for which the new image location intersects with the display device.

2) The method according to claim 1, wherein the user and the display device are on-board a vehicle and wherein the method comprises detecting a movement of the user and the display device by detecting a movement of the vehicle.

3) The method according to claim 2, wherein the distance from the user to the display device remains the first distance when the user and the display device move to a new location relative to the virtual screen.

4) The method according to claim 2, wherein the method comprises detecting the movement of the vehicle using one or more of gyroscopic sensors, accelerometer sensors, GPS signals, mapping information and/or control signals for controlling the motion of the vehicle.

5) The method according to claim 1, wherein the movement of the user and the display device includes a rotational and/or translational movement.

6) The method according to claim 1, wherein the image comprises a photograph, text content and/or a frame from a video.

7) The method according to claim 1, wherein the method comprises generating audio associated with the image when the image is displayed on the display device; and move a source location of the audio in accordance with the new image location when displaying the image on the display device at the new image location.

8) The method according to claim 1, wherein the method comprises adapting the display of the image on the display device when the movement of the user and the display device relative to the virtual screen is above a threshold value.

9) The method according to claim 8, wherein adapting the display of the image on the display device includes displaying a predetermined image to replace the image on the display device.

10) The method according to claim 9, wherein the predetermined image includes a blank image and/or an image with a predetermined property.

11) The method according to claim 8, wherein the method comprises generating audio associated with the image when the display of the image on the display device has been adapted.

12) The method according to claim 8, wherein when the movement of the user and the display device relative to the virtual screen remains above the threshold value for a predetermined amount of time, the method comprises moving the virtual screen to a new virtual screen location corresponding to the location defined by projecting a line from the new location of the user and the display device to a third location, the distance of the third location being the same as the second location.

13) The method according to claim 12, wherein the method comprises incrementally moving the virtual screen to the new virtual screen location over a second predetermined period of time.

14) The method according to claim 8, wherein the threshold value corresponds to a movement for which no portion of the new image location intersects with the display device.

15) The method according to claim 1, wherein when no portion of the new image location intersects the display device, the method comprises adjusting the new image location such that at least a predetermined portion of the image intersects the display device.

16) The method according to claim 15, further comprising adapting the image for display to indicate that the new image location has been adjusted.

17) An apparatus for alleviating motion sickness in a user viewing an image on a display, the apparatus comprising circuitry configured to:
display an image on a display device, the display device being a first distance from a user viewing the image on the display device;
generate a virtual screen corresponding to the display device by projecting a line from the user and the display device to a second location, the distance of the second location from the user being greater than the first distance;
detect a movement of the user and the display device to a new location relative to the virtual screen;
determine a new image location by projecting a line from the virtual screen location to the new location of the user;
and display an image on the display device at the new image location for at least a portion of the image for which the new image location intersects with the display device.

18) A non-transitory computer readable medium comprising computer executable instructions that, when the instructions are executed by a computer, cause the computer to perform a method of alleviating motion sickness in a user viewing an image on a display comprising:
displaying an image on a display device, the display device being a first distance from a user viewing the image on the display device;
generating a virtual screen corresponding to the display device by projecting a line from the user and the display device to a second location, the distance of the second location from the user being greater than the first distance;
detecting a movement of the user and the display device to a new location relative to the virtual screen;
determining a new image location by projecting a line from the virtual screen location to the new location of the user;
and displaying an image on the display device at the new image location for at least a portion of the image for which the new image location intersects with the display device.
